(19) United States
(12) Patent Application Publication
(10) Pub. No.: US 2025/0101484 A1
(43) Pub. Date: Mar. 27, 2025

(54) METHOD TO INCREASE DRUG SUBSTANCE YIELD

(71) Applicant: PharmaEssentia Corporation, Taipei (TW)

(72) Inventors: Ko-Chung Lin, Lexington, MA (US); Yu-Kuei Tsai, Taipei (TW); Ming-Pin Hsu, Taipei (TW)

(73) Assignee: PharmaEssentia Corporation, Taipei (TW)

(21) Appl. No.: 18/883,789

(22) Filed: Sep. 12, 2024

Related U.S. Application Data
(60) Provisional application No. 63/540,749, filed on Sep. 27, 2023.

(51) Int. Cl. C12P 21/02; C12M 1/34; C12M 1/36

(52) U.S. Cl. CPC C12P 21/02; C12M 41/26; C12M 41/32; C12M 41/34; C12M 41/42; C12M 41/48

(57) ABSTRACT

The present disclosure includes an improved method of making a protein. The steps and conditions were described and carried out with significantly improved protein yield, thereby reducing manufacturing cost.

### CROSS-REFERENCE TO RELATED APPLICATIONS

The present application claims the benefit under 35 U.S.C. 119 to U.S. Provisional patent application Ser. No. 63/540,749 filed on Sep. 27, 2023, the content of which is incorporated herein by reference in its entirety.

### TECHNICAL FIELD

The present disclosure relates in general to the field of protein drug substance yield.

### SEQUENCE LISTING

This application is being filed electronically via EFS-Web and includes an electronically submitted sequence listing in xml format in ST.26 format. The .xml file contains a sequence listing entitled “024_SEQ_LISTING.xml” created on Aug. 22, 2024, and having a size of 4,338 bytes and 8,196 bytes on disk. The sequence listing contained in this xml file is part of the specification and is herein incorporated by reference in its entirety.

### BACKGROUND

Without limiting the scope of the disclosure, its background is described in connection with methods that improve the production of inclusion body cell mass and/or protein yield for a protein of interest such as granulocyte colony stimulating factor (GCSF), human interferon alpha, human interferon beta, human interferon alpha 2b, Pro-hIFN-α2b, monoclonal antibodies, and/or any protein/intermediates thereof.

Biologicals (e.g., monoclonal antibodies, large proteins such as interferon, GCSF, etc.) in the pharmaceutical industry are known to be unpredictable to consistently make and manufacturer, due to the fact that most of the proteins or “large molecules” are naturally derived. The manufacturing process of these proteins typically involves fermentation of a host cell, followed by cell processing, purification, or some other chemical reaction to further modify the protein.

However, large scale productions of these proteins are typically difficult and resulting in low yield from the economical perspective.

Another challenge associated with the production of protein is in the process of fermentation and/or post-fermentation cell processing control since the protein typically needs to be made at a certain production volume/condition, and each batch's characteristic might be different, therefore so does the yield of the protein or cell mass.

Therefore, there is a need for a novel method for make these different proteins and/or intermediates in large quantity than what is currently known to one ordinary skilled in the art.

### SUMMARY

The present disclosure relates to methods to increase fermentation yield for protein production.

In an aspect, the present disclosure relates to a method to increase recombinant protein production in a prokaryote host cell, comprising performing one or more fed-batch recombinant protein fermentation. The fed-batch recombinant protein fermentation includes the steps of: carrying out seeding (time S₀) of a culture of prokaryote host cell engineered to inducibly express a recombinant protein and transferring the culture into one or more fermenters each containing a culture medium; measuring a dissolved oxygen (DO) level, an agitation rate, and a pH in the one or more fermenters; supplying a carbon source feeding solution to any of the one or more fermenters, individually, whenever condition(s) (i) and/or (ii) are met: (i) said DO level exceeds above about 35% to 45% and said agitation rate exceeds about 300 to 1,000 rpm; (ii) said agitation rate exceeds about 400 to 700 rpm and said pH exceeds about 7.0 to 7.4; and supplying a nitrogen source to any of the one or more fermenters in which induction of expression of the recombinant protein has been initiated, individually, at about I₀ (time at initiation of induction) or I₁ or both; optionally, supplying a nitrogen source to any of the one or more fermenters, individually, at one or more time points selected from about S₅, S₆, S₇, S₈, S₉ and S₁₀. In another embodiment, the dissolved oxygen (DO) level, agitation rate, and pH are continuously and/or periodically monitored at a given time interval. In an aspect, the method includes using a program-controlled mechanism to monitor DO, pH, agitation rate, and/or other parameters.

In an aspect, the present disclosure relates to a method to increase recombinant protein production in a prokaryote host cell, comprising performing one or more batch recombinant protein fermentation. The batch recombinant protein fermentation includes the steps of: carrying out seeding (time S₀) of a culture of prokaryote host cell engineered to inducibly express a recombinant protein and transferring the culture into one or more fermenters each containing a culture medium; measuring a dissolved oxygen (DO) level, an agitation rate, and a pH in the one or more fermenters; supplying a carbon source feeding solution to any of the one or more fermenters, individually, whenever condition(s) (i) and/or (ii) are met: (i) said DO level exceeds above about 35% to 45% and said agitation rate exceeds about 300 to 1,000 rpm; (ii) said agitation rate exceeds about 400 to 700 rpm and said pH exceeds about 7.0 to 7.4; and supplying a nitrogen source to any of the one or more fermenters in which induction of expression of the recombinant protein has been initiated, individually, at about I₀ (time at initiation of induction) or I₁ or both; optionally, supplying a nitrogen source to any of the one or more fermenters, individually, at one or more time points selected from about S₅, S₆, S₇, S₈, S₉ and S₁₀. In another embodiment, the dissolved oxygen (DO) level, agitation rate, and pH are continuously and/or periodically monitored at a given time interval. In an aspect, the method includes using a program-controlled mechanism to monitor DO, pH, agitation rate, and/or other parameters.

In one embodiment, in the method to increase recombinant protein production in a prokaryote host cell, in condition (i), said DO level exceeds above about 35% to 40% (e.g., about 35, 36, 37, 38, 39, or 40%) and said agitation rate exceeds about 300 to 700 rpm (e.g., about 300, 350, 400, 450, 500, 550, 600, 650, or 700 rpm), optionally about 300 to 500 rpm. In another embodiment, said DO level exceeds above about 35% to 40% (e.g., about 35, 36, 37, 38, 39, or 40%) and said agitation rate exceeds about 300 to 500 rpm, optionally, about 300 to 400 rpm, 350 to 450 rpm, or 350 to 500 rpm. In some embodiments, said DO level exceeds above about 35% and said agitation rate exceeds about 300 to 500 rpm. In another embodiment, said DO level exceeds above about 35% and said agitation rate exceeds about 300 to 400 rpm, about 400 to 500 rpm, about 350 to 400 rpm, about 400 to 450 rpm, about 450 to 500 rpm, about 300 rpm, about 350 rpm, about 400 rpm, about 450 rpm, or about 500 rpm. In one embodiment, the said DO level exceeds above about 40% and said agitation rate exceeds about 300 to 500 rpm. In yet another embodiment, said DO level exceeds above about 40% and said agitation rate exceeds about 300 to 400 rpm, about 400 to 500 rpm, about 350 to 400 rpm, about 400 to 450 rpm, about 450 to 500 rpm, about 300 rpm, about 350 rpm, about 400 rpm, about 450 rpm, or about 500 rpm.

In one embodiment, in the method to increase recombinant protein production in a prokaryote host cell, in condition (ii), said agitation rate exceeds about 500 to 600 rpm and said pH exceeds about 7.0 to 7.4 (e.g., about 7.0, 7.1, 7.2, 7.3 or 7.4); optionally, said pH exceeds about 7.0 to 7.2 (e.g., about 7.0, 7.1, or 7.2) and said agitation rate exceeds about 500 rpm, 550 rpm, or 600 rpm. In another embodiment, said agitation rate exceeds about 600 rpm and said pH exceeds about 7.0 to 7.2; optionally, said pH exceeds about 7.2. In yet another embodiment, said agitation rate exceeds about 500 to 600 rpm and said pH exceeds about 7.2 to 7.4; optionally, said pH exceeds about 7.4 and said agitation rate exceeds about 500 rpm, 550 rpm, or 600 rpm.

In one embodiment, in the method to increase recombinant protein production in a prokaryote host cell, the nitrogen source is supplied to the one or more fermenters, individually, at about I₀ and one or more time points selected from about S₅, S₆, S₇, S₈, S₉ and S₁₀ (e.g., S₅, S₆, S₇, S₈, S₉, S₁₀, S₅ and S₇, S₅ and S₈, S₆ and S₉, S₆ and S₁₀, S₅, S₇, and S₉, or S₆, S₇, and S₁₀); optionally, at about I₀ and one or more time points selected from about S₅, S₆, S₇, and S₈ (e.g., S₅, S₆, S₇, S₈, S₅ and S₇, S₅ and S₈, S₆ and S₈, S₆ and S₇, S₅, S₇, and S₈, or S₆, S₇, and S₈).

In another embodiment, in the method to increase recombinant protein production in a prokaryote host cell, the nitrogen source is supplied to the one or more fermenters, individually, at about I₁ and one or more time points selected from about S₅, S₆, S₇, S₈, S₉ and S₁₀ (e.g., S₅, S₆, S₇, S₈, S₉, S₁₀, S₅ and S₇, S₅ and S₈, S₆ and S₉, S₆ and S₁₀, S₅, S₇, and S₉, or S₆, S₇, and S₁₀); optionally, at about I₁ and one or more time points selected from about S₅, S₆, S₇, and S₈ (e.g., S₅, S₆, S₇, S₈, S₅ and S₇, S₅ and S₈, S₆ and S₈, S₆ and S₇, S₅, S₇, and S₈, or S₆, S₇, and S₈).

In yet another embodiment, in the method to increase recombinant protein production in a prokaryote host cell, the nitrogen source is supplied to the one or more fermenters, individually, at about I₀ and about I₁ and one or more time points selected from about S₅, S₆, S₇, S₈, S₉ and S₁₀ (e.g., S₅, S₆, S₇, S₈, S₉, S₁₀, S₅ and S₇, S₅ and S₈, S₆ and S₉, S₆ and S₁₀, S₅, S₇, and S₉, or S₆, S₇, and S₁₀); optionally, at about I₀ and about I₁ and at one or more time points selected from about S₅, S₆, S₇, and S₈ (e.g., S₅, S₆, S₇, S₈, S₅ and S₇, S₅ and S₈, S₆ and S₈, S₆ and S₇, S₅, S₇, and S₈, or S₆, S₇, and S₈).

In one embodiment, a carbon source feeding solution is supplied at least once to at least one fermenter during the fed-batch recombinant protein fermentation. In some embodiments, the carbon source feeding solution is supplied two or more separate times to at least one fermenter during the fed-batch recombinant protein fermentation. In one embodiment, the carbon source feeding solution is a glucose feeding solution, fructose feeding solution, galactose feeding solution, pyruvate feeding solution, or any combination thereof.

In one embodiment, in the method to increase recombinant protein production in a prokaryote host cell, the nitrogen source is supplied to the one or more fermenters, individually, at about I₀ and/or about I₁ and one or more time points selected from about S₅, S₆, S₇, S₈, S₉ and S₁₀ (e.g., S₅, S₆, S₇, S₈, S₉, S₁₀, S₅ and S₇, S₅ and S₈, S₆ and S₉, S₆ and S₁₀, S₅, S₇, and S₉, or S₆, S₇, and S₁₀); optionally, at about I₀ and/or about I₁ and one or more time points selected from about S₅, S₆, S₇, and S₈ (e.g., S₅, S₆, S₇, S₈, S₅ and S₇, S₅ and S₈, S₆ and S₈, S₆ and S₇, S₅, S₇, and S₈, or S₆, S₇, and S₈); and the carbon source feeding solution is supplied at least once or two or more separate times to the one or more fermenters during the fed-batch recombinant protein fermentation as triggered by either or both of conditions (i) and (ii). In an embodiment of the method, the nitrogen source can be supplied to the one or more fermenters, individually, at about I₀ or about I₁ and one or more time points selected from about S₅, S₆, S₇, and S₈ (e.g., S₅, S₆, S₇, S₈, S₅ and S₇, S₅ and S₈, S₆ and S₈, S₆ and S₇, S₅, S₇, and S₈, or S₆, S₇, and S₈); and the carbon source feeding solution can be supplied at least once or two or more separate times to the one or more fermenters during the fed-batch recombinant protein fermentation as triggered by either or both of conditions (i) and (ii).

In one embodiment, in the method to increase recombinant protein production in a prokaryote host cell, the carbon source feeding solution is a glucose feeding solution. In one embodiment, a cumulative total of about 800 to 1,500 g of glucose is supplied to at least one fermenter, per about 5 L fermenter capacity, by the end of the fed-batch recombinant protein fermentation. In some embodiments, a cumulative total of about 6,000 to 9,000 g of glucose is supplied to at least one fermenter by the end of the fed-batch recombinant protein fermentation, the at least one fermenter having about 40 L capacity.

In one embodiment, in the method to increase recombinant protein production in a prokaryote host cell, the glucose feeding solution supplied to the one or more fermenters, individually, includes D-(+)-Glucose; optionally, the glucose feeding solution supplied to the one or more fermenters, individually, contains about 600 to 900 g/L D-(+)-Glucose Anhydrous. In one embodiment, the glucose feeding solution supplied to the one or more fermenters, individually, contains H₃BO₃, CaCl₂·2H₂O, CuSO₄·5H₂O, MnSO₄·H₂O, (NH₄)₆MO₇O₂₄·4H₂O, EDTA, FeSO₄·7H₂O, ZnSO₄·7H₂O, 12N (37%) HCl, and/or MgSO₄·7H₂O; optionally, about 20 to 600 g/L MgSO₄·7H₂O. In another embodiment, the carbon source is added in a continuously fashion, or in a batch fashion, depending on the culture conditions such as dissolved oxygen (DO) level, an agitation rate, and a pH levels.

In one embodiment, in the method to increase recombinant protein production in a prokaryote host cell, the nitrogen source supplied to the one or more fermenters, individually, is a yeast extract, peptone, soytone, urea, tryptone, or any combinations thereof; optionally, the nitrogen source is a yeast extract solution. In some embodiments, the yeast extract solution supplied to the one or more fermenters, individually, has a concentration of about 100 to 600 g/L, 200 to 300 g/L, 300 to 400 g/L, 400 to 500 g/L, 500 to 600 g/L, 150 to 200 g/L, 200 to 250 g/L, 250 to 300 g/L, 300 to 350 g/L, 350 to 400 g/L, 400 to 450 g/L, 450 to 500 g/L, 100 g/L, 150 g/L, 200 g/L, 250 g/L, 300 g/L, 350 g/L, 400 g/L, 428 g/L, 450 g/L, 500 g/L, or 600 g/L. The total volume of nitrogen source can be adjusted based on fermentation scale.

In one embodiment, in the method to increase recombinant protein production in a prokaryote host cell, about 40 to 150 mL of the yeast extract solution is supplied to the one or more fermenters each time, individually; optionally about 300 to 1,500 mL of the yeast extract solution is supplied to the one or more fermenters each time, individually. In another embodiment, about 45 to 60 mL of the yeast extract solution is supplied to the one of more fermenters, individually, if supplied at any time point selected from about S₅, S₆, S₇, S₈, S₉ and S₁₀; and about 90 to 120 ml of the yeast extract solution is supplied to the one of more fermenters, individually, if supplied at about I₀ or I₁. In one embodiment, about 350 to 500 mL of the yeast extract solution is supplied to the one of more fermenters, individually, if supplied at any time point selected from about S₅, S₆, S₇, S₈, S₉ and S₁₀; and about 700 to 1,200 mL of the yeast extract solution is supplied to the one of more fermenters, individually, if supplied at about I₀ or I₁.

In some embodiments, in the method to increase recombinant protein production in a prokaryote host cell, the nitrogen source supplied to the one or more fermenters, individually, contains potassium, optionally in the form of K₂HPO₄ and/or KH₂PO₄; or wherein potassium, optionally in the form of K₂HPO₄ and/or KH₂PO₄, is supplied to the one or more fermenters, individually, at about the same time the nitrogen source is supplied. In one embodiment, the nitrogen source contains about 1.0 to 3.0 M of potassium, or wherein potassium is supplied to reach a final potassium concentration of about 35 to 40 mM in the one or more fermenters, individually.

In an embodiment of the method to increase recombinant protein production in a prokaryote host cell, the one or more fermenters, individually, can have about 5 L to 200 L or larger capacity (e.g., about 5 L, 10 L, 20 L, 30 L, 40 L, 50 L, 80 L, 100 L, 150 L, 200 L, or larger than 200 L); optionally, the one or more fermenters each has about a 5 L capacity; optionally, the one or more fermenters each has about a 40 L capacity; optionally, the one or more fermenters, individually, has 1 to 200 L of the medium.

In another embodiment, the concentration of each ingredient of the present disclosure can remain unchanged when the fermentation size is scaled upwards or downwards (e.g., about 5 L, 10 L, 20 L, 30 L, 40 L, 50 L, 80 L, 100 L, 150 L, 200 L, or larger than 200 L). In an aspect, the total volume of each ingredient, such as carbon and/or nitrogen source, may be adjusted depending on the fermentation size. Nonlimiting example include that for a 5 L to 40 L scale up, the volume required is eight times for 40 L fermentation as compared to 5 L. Additionally, or alternatively, customary adjustments on parameters are also contemplated in the present disclosure based on fermentation size.

In one embodiment, in the method to increase recombinant protein production in a prokaryote host cell, wherein said program-controlled mechanism for supplying a carbon source feeding solution includes one or more sensors which measure the DO level, pH, redox measurement, and/or carbon amount in the one or more fermenters, and wherein said program-controlled mechanism automatically injects the carbon source feeding solution into the one or more fermenters.

In one embodiment, in the method to increase recombinant protein production in a prokaryote host cell, the fed-batch recombinant protein fermentation is performed with an inoculum ratio of about 6.0 to 8.5%; optionally, about 6.5 to 7%, 7.0 to 7.5%, 7.5 to 8%, or 8.0 to 8.5%; optionally, about 6.25%, 6.3%, 6.4%, 6.5%, 6.67%, 6.7%, 6.8%, 6.95%, 7.0%, 7.24%, 7.3%, 7.33%, 7.48%, 7.5%, 7.6%, 7.76%, 7.8%, 7.9%, 8%, 8.14%, or 8.4%. In an embodiment of the method, the inoculum ratio can be about 6.0 to 8.5%; optionally, about 6.5 to 7%, 7.0 to 7.5%, 7.5 to 8%, or 8.0 to 8.5%; optionally, about 6.25%, 6.3%, 6.4%, 6.5%, 6.67%, 6.7%, 6.8%, 6.95%, 7.0%, 7.24%, 7.3%, 7.33%, 7.48%, 7.5%, 7.6%, 7.76%, 7.8%, 7.9%, 8%, 8.14%, or 8.4%.

In one embodiment, in the method to increase recombinant protein production in a prokaryote host cell, induction of expression of the recombinant protein is initiated when OD₆₀₀ in the culture in a fermenter reaches about 40 to 80, optionally, about 40 to 50, 50 to 60, 60 to 70, 70 to 80, 55 to 60, 60 to 65, 65 to 70, 70 to 75, 75 to 80, 40, 45, 49, 52, 55, 60, 66, 67, 68, 69, 70, 71, 72, 75, 78, or 80. In one embodiment of the method, expression of the recombinant protein is induced by adding Isopropyl-beta-D-1-thiogalactopyranoside (IPTG), optionally, about 3 to 5 mL of about 0.70 to 0.85 mM of IPTG.

In an embodiment of the method to increase recombinant protein production in a prokaryote host cell, the recombinant protein can be any of VX-001, AVE-9633, AC-9301, NY-ESO-1 vaccine, erythropoietin, EPO-Fc, CNTO-528, AMG-114, JR-013, Factor XIII, IFN-α2b, Pro-IFN-α2b, anti-PD1 antibody, and GCSF or a combination thereof.

In an embodiment of the method to increase recombinant protein production in a prokaryote host cell, the prokaryote host cell comprises an E. coli cell. In one embodiment, the E. coli is BL-21 strain, E. coli BLR-Codon Plus (DE3)-RIL, E. coli BLR-Codon Plus (DE3), or E. coli BL21-CodonPlus (DE3)-RIL.

In one embodiment, the method to increase recombinant protein production in a prokaryote host cell further includes a step of collecting the culture of E. coli host cell or a sample of the culture in the one or more fermenters after induction of expression of the recombinant protein, individually. In one embodiment, the method further comprising processing inclusion bodies in the collected culture of E. coli cell or sample to obtain a crude protein extract. In one embodiment, the step of inclusion body processing includes: resuspending the E. coli cells in the collected culture or sample in a buffer; homogenizing the E. coli cells one or more times at about 800 to 1,200 bar, thereby obtaining a cell mixture; centrifuging the cell mixture to form a cell pellet utilizing a batch centrifuging with a centrifugation speed about 20,000 to 30,000 rpm; alternatively, the centrifuge step can be carried our using a continuous centrifuging flow method with a feeding rate about 30-60 ml/min, and with a centrifugation speed about 20,000 to 30,000 rpm; collecting the cell pellet; and processing the cell pellet to obtain a crude protein extract. In an embodiment of the method, the culture or sample can be collected after or at about 14 to I₈ (e.g., after or at about I₄, I₅, I₆, I₇, or I₈). In one embodiment, the total time for the fed-batch recombinant protein fermentation includes about 10 to 95 hours or more, optionally about 14±1, 16±1, 24±1, 30±1, 36±1, 48±1, 60±1, 72±1, 84, ±1, or 96±1 hours.

In one embodiment, the method to increase recombinant protein production in a prokaryote host cell further includes determining yield of the expressed recombinant protein in the crude protein extract, wherein the yield is increased by at least about 5%, at least 10%, at least 15%, at least 20%, at least 25%, at least 30%, at least 35%, at least 40%, at least 45%, at least 50%, at least 55%, at least 60%, at least about 62.7%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 167%, at least 180%, at least 190%, at least 200%, at least 210%, at least 220%, at least 230%, at least 240%, at least 250%, at least 260%, at least 270%, at least 280%, at least 290%, at least 300%, at least 350%, at least 383%, at least 400%, at least 450%, at least 500%, at least 550%, at least 600%, at least 650%, at least 700%, at least 748%, at least 750%, at least 800%, at least 9 folds, at least 10 folds, at least 20 folds or more, as compared to another fermentation method. In one embodiment, the yield is determined for a fermenter having about a 40 L capacity and the yield is increased by at least about 100% to 200%, 100%, 105%, 110%, 115%, 120%, 125%, 130%, 135%, 140%, 145%, 150%, 155%, 160%, 165%, 170%, 175%, 180%, 190%, or 200%, as compared to the yield from a fermenter having about a 5 L capacity in the other fermentation method. In one embodiment, the recombinant protein is GCSF, Pro-IFN-α2b, and/or anti-PD1 monoclonal antibody.

In one embodiment, the method to increase recombinant protein production in a prokaryote host cell further includes determining yield of the expressed recombinant protein in the crude protein extract, wherein the yield is about 5 to 10 g of the recombinant protein in a fermenter having about a 5 L capacity; optionally, wherein the yield is about 20 to 30 g of the recombinant protein in a fermenter having about a 40 L capacity; optionally, the recombinant protein is GCSF, Pro-IFN-α2b, and/or anti-PD1 monoclonal antibody.

In one embodiment, the method to increase recombinant protein production in a prokaryote host cell further comprises the following steps performed before the fed-batch recombinant protein fermentation: obtaining a codon optimized nucleic acid encoding the recombinant protein; transfecting said codon optimized nucleic acid into the prokaryotic host cell; and polarizing said prokaryotic host cell and culturing said prokaryotic host cell at about 36-370 C overnight or about 12-18 hours, thereby resulting in a whole cell broth which includes the transfected codon optimized nucleic acid.

In another aspect, described herein is a program-controlled mechanism or equipment comprising: (a) an apparatus equipped with one or more sensors capable of measuring the oxygen level, pH, and/or agitation of culture medium in a fermentation reactor in a fermentation process; and (b) a feeding solution injection system integrated within said program-controlled mechanism that is programmed to provide one or more feeding solutions to a fermentation reactor based on measured oxygen levels, pH and/or agitation in the culture medium. In an aspect, the mechanism or equipment can measure and/or feed solution for a periodical interval (e.g., every 30 minutes for up to 96 hours or longer) or in a continuous fashion.

In one embodiment of the program-controlled mechanism or equipment, the one or more sensors transmit real-time measurements to the program-controlled mechanism; optionally, the program-controlled mechanism or equipment further comprises a control unit configured to receive sensor measurements and automatically adjust the injection rate and volume of the feeding solution based on the measured parameters. In another embodiment, the program-controlled mechanism or equipment further comprises a control unit configured to receive sensor measurements and automatically adjust the injection rate and volume of the feeding solution based on the measured parameters. In one embodiment, feeding solution injection system comprises a reservoir, a metering pump, and a distribution system to deliver the feeding solution into the fermentation process; optionally, the feeding solution injection system is programed to include predetermined setpoints and control algorithms to modify glucose levels, dissolved oxygen, and/or pH within the fermentation process in the culture medium.

In yet another aspect, this disclosure includes a method for automatically regulating glucose concentration, oxygen level, pH and/or agitation during fermentation, comprising: (a) measuring parameters of oxygen level, pH and/or agitation of the fermentation with one or more sensors; (b) transmitting the measured parameters to a program-controlled mechanism; and (c) automatically adjusting the injection rate, volume of a feeding solution and agitation based on the measured parameters. In another aspect, the method for measuring, transmitting, and/or adjusting injection rate can be performed in a periodical fashion (e.g., every 30 minutes for up to 96 hours, or longer), or in a continuous fashion.

### BRIEF DESCRIPTION OF THE DRAWINGS

For a more complete understanding of the features and advantages of the present disclosure, reference is now made to the detailed description herein along with the accompanying figures and in which:

FIG. 1 shows a structure referred to as proline-IFNα2b or also known as Pro-hIFN alpha 2b

FIG. 2 shows an image of an SDS-PAGE of Pro-hIFN alpha 2b at various stages of post IPTG induction using a manufacturing method disclosed in the present disclosure. The arrow denotes the size and expected band location. Lane M: Marker; Lane RS: Pro-hIFN alpha 2b drug reference standard (2 μg); Lane 0: initial time Isopropyl-beta-D-1-thiogalactopyranoside (IPTG) used for protein induction at time=0 hr; Lane 1: IPTG post initial induction time at about 1.0 hr; Lane 2: IPTG post initial induction time at about 2.0 hr; Lane 3: IPTG post initial induction time at about 3.0 hr, Lane 4: IPTG post initial induction time at about 4.0 hr; Lane 5: IPTG post initial induction time at about 5.0 hr. Y axis denotes size.

FIG. 3 shows an image of an SDS-PAGE performed on protein Pro-hIFN alpha 2b after purification. The arrow denotes the size and expected band location. M stands for Marker, RS stands for Pro-hIFN alpha 2b drug reference standard (2 μg), and SA stands for purified protein sample (5.0 μg loading).

FIG. 4 shows a HPLC graph on purity of Pro-hIFN alpha 2b using a fermentation method as disclosed in the present disclosure. Peak 1 denotes for Pro-hIFN alpha 2b. Peak 2 is solvent peak. X-axis is retention time in minutes. Y-axis denotes amount.

FIG. 5 denotes an amino acid sequence of GCSF with Methionine at the N terminus.

FIG. 6 denotes an amino acid sequence of GCSF.

FIG. 7 denotes the amino acid sequence of an anti-PD-1 monoclonal antibody.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As use herein, all numeric ranges are inclusive of the numbers defining the range. The headings provided herein are not limitations of the various aspects of the disclosure, which can be had by reference to the specification as a whole.

Throughout this disclosure, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range and can be up to two decimals for such number. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In addition, the term “about” refers to a value or composition that is within an error range for the particular reference value or composition as determined by one of ordinary skill in the art, which will depend in part on how the value or composition is measured or determined, i.e., the limitations of the measurement system. For example, “about” can mean within 1, or more than 1 standard deviation per the practice in the art. Alternatively or additionally, “about” can mean a range of up to ±5%, 10%, 15%, 20 and/or 25% of a particular reference value. For example, in the context of a ratio, the meaning “about” for a reference value of 8.14 include, 8.55, 8.95, 9.36, 9.77, 10.18, as well as 7.73, 7.33, 6.92, 6.51, and 6.11. Furthermore, the terms can also mean up to an order of magnitude or up to three (3) fold ± of a reference value, depending on the context known by one skilled in the art. For example, about 6.0 M of IPTG can range from 2.0 M to 18.0 M of IPTG. When particular values or compositions are provided in the application and claims, unless otherwise stated, the meaning of “about” should be construed to be within an acceptable error range for that particular value or composition.

As used herein, the term “protein” or “protein of interest” is used to include any protein (either natural or recombinant), present in a mixture, for which manufacturing in high volume and/or purification in is desired. Such proteins of interest include, without limitation, human or mouse interferon alpha, beta and gamma, enzymes, hormones, growth factors, cytokines, immunoglobulins (e.g., monoclonal antibodies), and/or any fusion proteins, and derivatives and portions thereof.

The terms “cell density”, “viable cell density”, and “cell concentration,” as used herein, refer interchangeably to the number of metabolically active cells per unit volume of a cell culture.

As used herein, the term “inoculum ratio” can refer to the ratio of “S/I”, which denotes the substrate to inoculum ratio (S/I) expressed in percentages. This ratio can be a factor that affects not only the stability of the fermentation but also the capacity of the substrate, which controls fermentation of a host cell as compared to its yield capacity based on environmental factors. Inoculum preparation involves obtaining the organisms in a state that is compatible with inoculation into cell culture, tissue culture, media, and fermenters. It is usually to achieve a level of viable biomass in a suitable physiological state for use as an inoculum. This has application in industrial microbiology for obtaining products such as antimicrobials, enzymes, beverages, drugs, toxins, vitamins, amino acids, organic acids, solvents, food products, and recombinant proteins. An inoculum is typically at an active growth stage and size, free from contamination, and have product-forming ability. Culture and production medium condition are needed for providing the right environment for inoculum. Inoculum quality can be enhanced by strain improvement and cell immobilization technology.

The term “cell bank” as used herein, refers to a storage of biological cells.

The terms “bioreactor” and “fermenter” can be used interchangeably herein to refer to any vessel or other means of producing and maintaining a biological cell culture including, but not limited to, a perfusion/perfused bioreactor. A bioreactor or fermenter can be used to perform a fermentation process, including but not limited to a fed-batch fermentation. A fermenter may be equipped with sensors and mechanisms to automatically or manually monitor and control the conditions within the fermenter (e.g., oxygen level, pH level, and agitation rate) during the fermentation process. A fermenter may also be equipped with mechanisms to automatically or manually remove or inject cells, culture media, nutrients, or any compounds or materials during the fermentation process, and can be in sizes for small (lab based) to large scale (commercial) production.

The terms “perfusion” or “cyto-perfusion” as used herein, refer to a fermentation or cell culture process used to produce a targeted biological product, e.g., an antibody or recombinant protein, in which a high concentration of cells within a sterile chamber receives fresh growth medium continually as the spent medium which may contain a targeted biological product that is harvested.

In the context of host cell fermentation, the capital letter “I” denotes induction of protein in a host cell, whereby introducing a chemical, typically IPTG, can induce the host cell to express the protein that was cloned or transfected. The letter can be followed with a numeric subscript indicating the time in hour with respect to induction, for example I₀, which refers to the time of initial induction. I₁ and I₂ denote about one hour and two hours after initial induction, respectively. Similarly, in the same context, the capital letter “S” denotes initial culture seeding in order to grow host cell in a fermentation media. The letter can be followed with a numeric subscript indicating the time in hour with respect to initial seeding, for example S₀, which refers to the time point of initial seeding. S₁ and S₂ denote about one hour and two hours after initial seeding, respectively.

The term “fed-batch fermentation” as used herein, refers to a bioreactor operational state in which the volume of the biological cell culture is adjusted by introducing a cell growth medium into the bioreactor. It is a combination of batch and continuous mode where substrate is added into the reactor without removing the medium to avoid the problem of substrate inhibition. It is distinguishable from batch fermentation by the addition, during operations, of a certain amount of fresh substrate and by the consequent withdrawal of a proportioned amount of broth.

The term “perfused centrifugation” as used herein, refers to a bioreactor operational state in which a perfusion device is operated to retain cells within the bioreactor while a cell growth medium is introduced into the bioreactor and a spent medium is harvested from the bioreactor, while maintaining the volume of the cell culture at a substantially constant level.

The term “fed-batch centrifugation” as used herein, refers to a bioreactor operational state in which the volume of the biological cell culture is adjusted by introducing a cell growth medium into the bioreactor. It is a combination of batch and continuous mode where substrate is added into the reactor without removing the medium to avoid the problem of substrate inhibition. It is distinguishable from batch fermentation by the addition, during operations, of a certain amount of fresh substrate and by the consequent withdrawal of a proportional amount of broth.

The term “perfused batch state” as used herein, refers to a bioreactor operational state in which a perfusion device is operated to retain cells within the bioreactor while a cell growth medium is introduced into the bioreactor and a spent medium is harvested from the bioreactor, while maintaining the volume of the cell culture at a substantially constant level.

The term “perfused fed-batch state” as used herein, refers to a bioreactor operational state in which a perfusion device is operated to retain cells within the bioreactor while a cell growth medium is introduced into the bioreactor and a spent medium is harvested from the bioreactor, while maintaining the cell concentration at a substantially constant level.

As used herein, the acronym “Y.E.” refers to yeast extract, wherein the non-animal yeast extract can be derived naturally for the purpose of fermentation. The term “Y.E. feeding strategy” refers to the timing to add yeast extract in a fermentation process.

As used herein, the terms “purifying,” “separating,” or “isolating,” are used interchangeably. The terms refer to increasing the degree of purity of a protein of interest from a composition or sample comprising the protein of interest and one or more impurities. Typically, the degree of purity of the protein of interest is increased by removing (completely or partially) at least one impurity from the composition. As one skilled in the art knows, the more steps of purification, yield will decrease (or suffer), and the amount of yield drop will depend on the amount of impurity and purification methods. In a non-liming example, the meaning of purifying includes, but not limited to ionic charged chromatography, which can be anionic or cationic.

As used herein, the term “buffered”, or “buffer” denotes a solution in which changes of pH due to the addition or release of acidic or basic substances is leveled by a buffer substance. Any buffer substance resulting in such an effect can be used. In some embodiments, pharmaceutically acceptable buffer substances are used, such as, e.g., phosphoric acid or salts thereof, acetic acid or salts thereof, citric acid or salts thereof, morpholine, 2-(N-morpholino) ethanesulfonic acid or salts thereof, histidine or salts thereof, glycine or salts thereof, or Tris(hydroxymethyl)aminomethane (TRIS) or salts thereof. In one embodiment, phosphoric acid or salts thereof, or acetic acid or salts thereof, or citric acid or salts thereof, or histidine or salts thereof are used as the buffer substance. Optionally the buffered solution can comprise an additional salt, such as, e.g., sodium chloride, sodium sulphate, potassium chloride, potassium sulfate, sodium citrate, or potassium citrate. Other buffers known in the art, such as TEN buffer, can also be used.

The term “medium” or “media” generally refer to a cell culture medium for maintaining cells or allowing cell growth, which may comprise salts, amino acids, vitamins, lipids, detergents, buffers, growth factors, hormones, cytokines, trace elements and/or carbohydrates. Examples of salts include magnesium salts, for example MgCl₂×6H₂O; iron salts, for example FeSO₄×7H₂O; potassium salts, for example KH₂PO₄, KCl; sodium salts, for example NaH₂PO₄ or Na₂HPO₄; and calcium salts, for example CaCl₂×2H₂O. Examples of amino acids include all 20 known proteinogenic amino acids, for example histidine, glutamine, threonine, serine, methionine. Examples of vitamins include ascorbate, biotin, choline, myo-inositol, and D-panthothenate, riboflavin. Examples of lipids include fatty acids, for example linoleic acid and oleic acid; and soy peptone and ethanol amine. Examples of detergents include Tween 80 and Pluronic F68. An example of a buffer is HEPES. Examples of growth factors/hormones/cytokines include IGF, hydrocortisone, and recombinant insulin. Examples of trace elements are known to a person skilled in the art and include Zn, Mg and Se. Examples of carbohydrates include glucose, fructose, galactose and pyruvate. Other buffers known in the art can also be used depending on host cell and fermentation process; one example is SYN media known in the art.

As used herein, the term “polypeptide” is interchangeably used with “protein” and refers to any chain or chains of two or more amino acids and does not refer to a specific length of the product. The term “protein” encompasses a molecule comprised of one or more polypeptides, which can in some instances be associated by bonds other than amide bonds. On the other hand, a protein can also be a single polypeptide chain. In this latter case, the single polypeptide chain can in some instances comprise two or more polypeptide subunits fused together to form a protein. The terms “polypeptide” and “protein” can also refer to a product of post-expression modifications, including without limitation glycosylation, phosphorylation, derivatization by known protecting/blocking groups, proteolytic cleavage, or modification by non-naturally occurring amino acids. A polypeptide or protein can be derived from a natural biological source or produced by recombinant technology but is not necessarily translated from a designated nucleic acid sequence. It can be generated in any manner, including by chemical synthesis.

As used herein, the term “polarization” as used in biology is a process or act of producing positive and negative electric charge values to opposite ends, such as the electrical charge inside a cell relative to the surrounding environment. In an embodiment, polarization of cells can mobilize intracellular protein to signal the cell to synthesize a gene of interest.

As used herein, the term “isoelectric point” or “pi” of a protein refers to a measure of the pH of a solution in which a protein carries no net charge. When a protein is found at a pH equivalent to its pi, it will carry globally neutral net electric charge. Proteins that have a pi lower than the pH of its solution will carry a net negative charge. Likewise, proteins that have a pi higher than the pH of its solution will carry a net positive charge.

As used herein, the term “loading buffer” refers to the buffer used to prepare and load a mixture or sample into the chromatography unit.

The term “vector” refers to a carrier nucleic acid molecule into which a nucleic acid sequence can be inserted for introduction into a host cell. Vectors capable of directing the expression of nucleic acids to which they are operatively linked are referred to herein as “expression vectors”. Vectors can be viral vectors or non-viral vectors.

As used herein, the term “host cell” refers to a cell to which a vector or expression vector can be or has been introduced. A host cell can contain an expression vector for expressing a nucleic acid encoding a protein of interest in the host cell. Some examples include Bacillus, yeast from the genus of Saccharomyces, Pichia, Aspergillus, Fusarium, Kluyveromyces, CHO (Chinese Hamster Ovary) cell, hybridomas, BHK (Baby Hamster Kidney) cell, myeloma cell, HEK-293 cell, human lymphoblastoid cell and mouse cell, for example a NSO cell. Other examples include E. coli (with various modifications available to one skilled in the art), including but not limited to E. coli BL-21, E. coli BLR-Codon Plus (DE3)-RIL, E. coli BLR-Codon Plus (DE3), E. coli BLR-(DE3), or E. coli BL21-CodonPlus (DE3)-RIL, all known to one skilled in the art.

As used herein, the term “anti PD-1 antibody” or “anti PD-1 monoclonal antibody” are used interchangeably. Anti PD-1 antibody includes a humanized immunoglobulin (Ig) G4 monoclonal antibody directed against the negative immunoregulatory human cell surface receptor programmed cell death 1 (PD-1), acting as immune checkpoint inhibitory and antineoplastic activities.

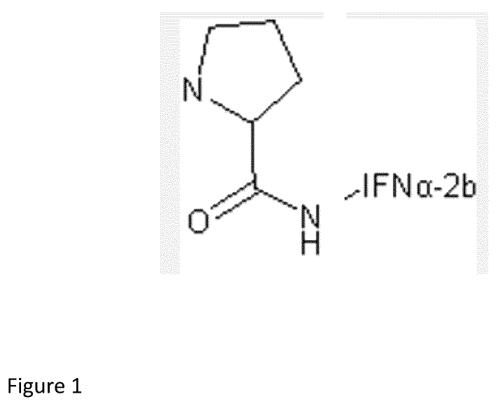

As used herein, the term “Pro-hIFN alpha 2b” refers to chemical formula as shown in FIG. 1, wherein human interferon alpha 2b contains an extra proline at the N terminus. Details of the molecule can also be found in U.S. Pat. No. 8,106,160 B2, the content of which is incorporated by reference herein in its entirety.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers or excipients are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, perfuming agents, preservatives and antioxidants can also be present in the composition, according to the judgment of the formulator.

Scientifically, it is known that increasing the number of steps and/or adding extra ingredients can cause yield decrease in protein manufacturing by lowering the total final product yield. For example, even if a stepwise yield for each manufacturing process can be as high as 90.0%, after 10 steps, the yield calculation is 0.9 multiply by itself 10 times which leads to overall yield of 34%. In other words, the more steps required, yield suffers. Nonetheless, the present disclosure surprising and unexpectedly demonstrates the opposite effect of the above known scientific concept. Utilizing the present disclosed method, the method increases the total number of steps, including, but not limited to adding steps relating to yeast extract and/or extra glucose feeding, yet no yield drop observed and the opposite effect of yield increase is shown. Moreover, the changes do not incrementally increase yield like routine optimization, but surprisingly and unexpectedly increase protein yield by, for example, at least about 113.5%, 126.5%, 150%, 162.7%, 180%, 213%, 300%, 325%, 350%, 383%, 400% or more as compared to method known in the art after the step of fermenting and inclusion body (IB) processing to generate a crude protein extract. Additionally, or alternatively, the yield increase can be seen in post fermentation process before inclusion body processing, where the yield of the crude protein can increase by at least about 113.5, 126.5, 150, 162.7, 180, 213%, 300%, 325%, 350%, 383%, 400% or more. The increase and/or improvements observed can be defined relative to the yield of a similar or substantially similar method that essentially differs from a method described herein only in the steps of controlling feeding the culture or fermentation reactor with a feeding solution based on dissolved oxygen content, agitation rate and/or pH, as discussed herein. Alternatively, or additionally, the increase and/or improvements observed can be defined as compared to the method disclosed in U.S. Pat. No. 8,106,160 B2.

The biopharmaceuticals (or protein) of the present disclosure where its manufacturing yield can be increased include, but not limited to: BOTOX, Myobloc, Neurobloc, Dysport (or other serotypes of botulinum neurotoxins), anti-PD1 monoclonal antibody, alglucosidase alpha, daptomycin, YH-16, choriogonadotropin alpha, filgrastim, cetrorelix, interleukin-2, aldesleukin, teceleulin, denileukin diftitox, interferon alpha-n3 (injection), interferon alpha-n1, DL-8234, interferon, Suntory (gamma-la), interferon gamma, thymosin alpha 1, tasonermin, DigiFab, ViperaTAb, EchiTAb, CroFab, nesiritide, abatacept, alefacept, Rebif, eptoterminalfa, teriparatide (osteoporosis), calcitonin injectable (bone disease), calcitonin (nasal, osteoporosis), etanercept, hemoglobin glutamer 250 (bovine), drotrecogin alpha, collagenase, carperitide, recombinant human epidermal growth factor (topical gel, wound healing), DWP401, darbepoetin alpha, epoetin omega, epoetin beta, epoetin alpha, desirudin, lepirudin, bivalirudin, nonacog alpha, Mononine, eptacog alpha (activated), recombinant Factor VIII+VWF, Recombinate, recombinant Factor VIII, Factor VIII (recombinant), Alphnmate, octocog alpha, Factor VIII, palifermin, Indikinase, 19eticuloses19, alteplase, pamiteplase, reteplase, nateplase, monteplase, follitropin alpha, rFSH, hpFSH, micafungin, pegfilgrastim, lenograstim, nartograstim, sermorelin, glucagon, exenatide, pramlintide, iniglucerase, galsulfase, Leucotropin, molgramostirn, triptorelin acetate, histrelin (subcutaneous implant, Hydron), deslorelin, histrelin, nafarelin, leuprolide sustained release depot (ATRIGEL), leuprolide implant (DUROS), goserelin, Eutropin, KP-102 program, somatropin, mecasermin (growth failure), enfuvirtide, Org-33408, insulin glargine, insulin glulisine, insulin (inhaled), insulin lispro, insulin deternir, insulin (buccal, RapidMist), mecasermin rinfabate, anakinra, celmoleukin, 99 mTc-apcitide injection, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex' recombinant human luteinizing hormone, dornase alpha, trafermin, ziconotide, taltirelin, diboterminalfa, atosiban, becaplermin, eptifibatide, Zemaira, CTC-111, Shanvac-B, HPV vaccine (quadrivalent), octreotide, lanreotide, ancestirn, agalsidase beta, agalsidase alpha, laronidase, prezatide copper acetate (topical gel), rasburicase, ranibizumab, Actimmune, PEG-Intron, Tricomin, recombinant house dust mite allergy desensitization injection, recombinant human parathyroid hormone (PTH) 1-84 (sc, osteoporosis), epoetin delta, transgenic antithrombin III, Granditropin, Vitrase, recombinant insulin, interferon-alpha (oral lozenge), GEM-21S, vapreotide, idursulfase, omnapatrilat, recombinant serum albumin, certolizumab pegol, glucarpidase, human recombinant CI esterase inhibitor (angioedema), lanoteplase, recombinant human growth hormone, enfuvirtide (needle-free injection, Biojector 2,000), VGV-1, interferon (alpha), lucinactant, aviptadil (inhaled, pulmonary disease), icatibant, ecallantide, omiganan, Aurograb, pexigananacetate, ADI-PEG-20, LDI-200, degarelix, cintredelinbesudotox, Favld, MDX-1379, 1SAtx-247, liraglutide, teriparatide (osteoporosis), tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone (sustained release injection), recombinant G-CSF, insulin (inhaled, AIR), insulin (inhaled, Technosphere), insulin (inhaled, AERx), RGN-303, DiaPep277, interferon beta (hepatitis C viral infection (HCV)), interferon alpha-n3 (oral), belatacept, transdermal insulin patches, AMG-531, MBP-8298, Xerecept, opebacan, AIDSVAX, GV-1001, LymphoScan, ranpirnase, Lipoxysan, lusupultide, MP52 (beta-tricalciumphosphate carrier, bone regeneration), melanoma vaccine, sipuleucel-T, CTP-37, Insegia, vitespen, human thrombin (frozen, surgical bleeding), thrombin, TransMID, alfimeprase, Puricase, terlipressin (intravenous, hepatorenal syndrome), EUR-1008M, recombinant FGF-I (injectable, vascular disease), BDM-E, rotigaptide, ETC-216, P-113, MBI-594AN, duramycin (inhaled, cystic fibrosis), SCV-07, OPI-45, Endostatin, Angiostatin, ABT-510, Bowman Birk Inhibitor Concentrate, XMP-629, 99 mTc-Hynic-Annexin V, kahalalide F, CTCE-9908, teverelix (extended release), ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, ovarian cancer immunotherapeutic vaccine, SB-249553, Oncovax-CL, OncoVax-P, BLP-25, CerVax-16, multi-epitope peptide melanoma vaccine (MART-I, gp100, tyrosinase), nemifitide, rAAT (inhaled), rAAT (dermatological), CGRP (inhaled, asthma), pegsunercept, thymosinbeta4, plitidepsin, GTP-200, ramoplanin, GRASPA, OBI-1, AC-100, salmon calcitonin (oral, eligen), calcitonin (oral, osteoporosis), examorelin, capromorelin, Cardeva, velafermin, 131I-TM-601, KK-220, T-10, ularitide, depelestat, hematide, Chrysalin (topical), rNAPc2, recombinant Factor V111 (PEGylated liposomal), bFGF, PEGylated recombinant staphylokinase variant, V-10153, SonoLysis Prolyse, NeuroVax, CZEN-002, islet cell neogenesis therapy, rGLP-1, BIM-51077, LY-548806, exenatide (controlled release, Medisorb), AVE-0010, GA-GCB, avorelin, ACM-9604, CETi-1, Hemospan, VAL (injectable), fast-acting insulin (injectable, Viadel), intranasal insulin, insulin (inhaled), insulin (oral, eligen), recombinant methionyl human leptin, Pro-hIFN alpha 2b, pitrakinra subcutaneous injection, eczema), pitrakinra (inhaled dry powder, asthma), Multikine, RG-1068, MM-093, NBI-6024, AT-001, PI-0824, Org-39141, Cpn10 (autoimmune diseases/inflammation), talactoferrin (topical), rEV-131 (ophthalmic), rEV-131 (respiratory disease), oral recombinant human insulin (diabetes), RPI-78 M, oprelvekin (oral), CYT-99007 CTLA4-Ig, DTY-001, valategrast, interferon alpha-n3 (topical), IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, malaria vaccine (virosomes, PeviPRO), ALTU-135, parvovirus B19 vaccine, influenza vaccine (recombinant neuraminidase), malaria/HBV vaccine, anthrax vaccine, Vacc-5q, Vacc-4x, HIV vaccine (oral), HPV vaccine, Tat Toxoid, YSPSL, CHS-13340, PTH (1-34) liposomal cream (Novasome), Ostabolin-C, PTH analog (topical, psoriasis), MBRI-93.02, MTB72F vaccine (tuberculosis), MVA-Ag85A vaccine (tuberculosis), FARA04, BA-210, recombinant plague FIV vaccine, AG-702, OxSODrol, rBetV1, Der-p1/Der-p2/Der-p7 allergen-targeting vaccine (dust mite allergy), PR1 peptide antigen (leukemia), mutant ras vaccine, HPV-16 E7 lipopeptide vaccine, labyrinthin vaccine (adenocarcinoma), CML vaccine, WT1-peptide vaccine (cancer), IDD-5, CDX-110, Pentrys, Norelin, CytoFab, P-9808, VT-111, icrocaptide, telbermin (dermatological, diabetic foot ulcer), rupintrivir, 21eticuloses, rGRF, HA, alpha-galactosidase A, ACE-011, ALTU-140, CGX-1160, angiotensin therapeutic vaccine, D-4F, ETC-642, APP-018, rhMBL, SCV-07 (oral, tuberculosis), DRF-7295, ABT-828, ErbB2-specific immunotoxin (anticancer), DT3SSIL-3, TST-10088, PRO-1762, Combotox, cholecystokinin-B/gastrin-receptor binding peptides, 1111n-hEGF, AE-37, trasnizumab-DM1, Antagonist G, IL-12 (recombinant), PM-02734, IMP-321, rhIGF-BP3, BLX-883, CUV-1647 (topical), L-19 based radio immunotherapeutics (cancer), Re-188-P-2045, AMG-386, DC/1540/KLH vaccine (cancer), VX-001, AVE-9633, AC-9301, NY-ESO-1 vaccine (peptides), NA17.A2 peptides, melanoma vaccine (pulsed antigen therapeutic), prostate cancer vaccine, CBP-501, recombinant human lactoferrin (dry eye), FX-06, AP-214, WAP-8294A (injectable), ACP-HIP, SUN-11031, peptide YY [3-36] (obesity, intranasal), FGLL, atacicept, BR3-Fc, BN-003, BA-058, human parathyroid hormone 1-34 (nasal, osteoporosis), F-18-CCR1, AT-1,100 (celiac disease/diabetes), JPD-003, PTH (7-34) liposomal cream (Novasome), duramycin (ophthalmic, dry eye), CAB-2, CTCE-0214, erythropoietin, EPO-Fc, CNTO-528, AMG-114, JR-013, Factor XIII, aminocandin, PN-951, 716155, SUN-E7001, TH-0318, BAY-73-7977, teverelix (immediate release), EP-51216, hGH (controlled release, Biosphere), OGP-I, sifuvirtide, TV4710, ALG-889, Org-41259, rhCC10, F-991, thymopentin (pulmonary diseases), r (m) CRP, hepatoselective insulin, subalin, L19-IL-2 fusion protein, elafin, NMK-150, ALTU-139, EN-122004, rhTPO, thrombopoietin receptor agonist (thrombocytopenia disorders), AL-108, AL-208, nerve growth factor antagonists (pain), SLV-317, CGX-1007, INNO-105, oral teriparatide (eligen), GEM-OSI, AC-162352, PRX-302, LFn-p24 fusion vaccine (Therapore), EP-1043, S. pneumoniae pediatric vaccine, malaria vaccine, Neisseria meningitidis Group B vaccine, neonatal group B streptococcal vaccine, anthrax vaccine, HCV vaccine (gpE1+gpE2+MF-59), otitis media therapy, HCV vaccine (core antigen+ISCOMATRIX), hPTH (1-34) (transdermal, ViaDerm), 768974, SYN-101, PGN-0052, aviscumnine, BIM-23190, tuberculosis vaccine, multi-epitope tyrosinase peptide, cancer vaccine, enkastim, APC-8024, GI-5005, ACC-001, TTS-CD3, vascular-targeted TNF (solid tumors), desmopressin (buccal controlled-release), onercept, TP-9201, granulocyte colony-stimulating factor (GCSF), and all versions of known interferons (INF), for example: Type I, Type II, or Type III IFNs, which can include IFNα, -β, -ω. -λ and -τ and subsequently chemically modified versions.

One embodiment in the present disclosure is use of the methods disclosed to manufacture pharmaceutical compositions including fermented protein (e.g., proline-IFNα2b); or for making protein GCSF, which can be modified into PEGylated-GCSF, in high quantities. In one embodiment, the end modified product produced by these methods can be purified, formulated by pharmaceutically acceptable carrier. These compositions can be administered to a subject parenterally, topically, or via an implanted reservoir. The term parenteral as used herein can include subcutaneous, intracutaneous, intravenous, intramuscular, intraarticular, intraarterial, intrasynovial, intrasternal, intrathecal, intralesional and intracranial injection or infusion techniques. In an embodiment, the composition is injected into a disfiguring tissue.

In some embodiments, the protein can include monoclonal antibody such as adalimumab (HUMIRA), infliximab (REMICADE, trademarked), rituximab (RITUXAN trademarked/MAB THERA trademarked) etanercept (ENBREL, trademarked), bevacizumab (AVASTIN, trademarked), trastuzumab (HERCEPTIN, trademarked), and/or any combinations in any quantity proportion thereof, or any other suitable polypeptide including biosimilars, interchangeables, and/or biobetters.

Other proteins are those listed below and in Table 1 of U.S. Patent Publication No. 2016/0097074 which is incorporated herein by reference in its entirety.

Any of the above proteins, for example, granulocyte colony-stimulating factor (GCSF), can be subsequently chemically modified; and additionally or alternatively, so can all versions of known interferons (INF), for example: Type I, Type II, or Type III IFNs, which can include IFNα, -β, -ω. -λ and -τ. In some embodiments, the present disclosure's methods can be used to increase fermentation, inclusion body cell mass, crude protein and/or purified protein, thereby increasing the final overall yield of interferon alpha 2b, interferon alpha, interferon beta, and/or interferon gamma.

In an embodiment, the present disclosure includes use of cultured cells utilized to produce other therapeutic proteins, e.g., antibodies, such as monoclonal antibodies, and/or recombinant proteins. In some embodiments, the cultured cells produce peptides, amino acids, fatty acids, or useful biochemical intermediates or metabolites. For example, in some embodiments, molecules having a molecular weight of about 300, 400, or 500 daltons to greater than about 100,000, 120,000, or 140,000 daltons can be produced. In embodiments, these molecules can have a range of complexity and can include posttranslational modifications including glycosylation.

The method of producing a recombinant protein typically starts with obtaining a host cell, introducing into the host cell a vector that can direct expression of a nucleic acid encoding the protein of interest in the host cell, and fermenting such host cell. The procedure typically uses a host cell that has been processed and stored into a cell bank. In an embodiment, the host cell that can be used include, but are not limited to: E. Coli competent cells such as E. coli BLR-Codon Plus (DE3)-RIL, E. coli BLR (DE3), E. coli BLR-Codon Plus (DE3), and/or E. coli BL21-CodonPlus (DE3)-RIL, or any suitable cells that are available. In another embodiment, these host cells in cell bank can be already engineered to express proteins of interest.

In some embodiments, a quantity about 1-50 μL (e.g., about 5, 10, 15, 20, 25, or 30 μL) of a host cell culture of interest (e.g., host cell capable of expressing a protein of interest) can be transferred to a small volumetric container already containing about 100-250 mL of medium or about 0.5 L, 1.0 L, 1.5 L, 2.0 L, 2.5 L, or 5 L of medium for production. A non-limiting example of media is SYN media known in the art. In one embodiment, the media is SYN media already premixed with about 10 to 50 μg/ml kanamycin and/or about 10 to 50 μg/ml chloramphenicol, which can be placed in a 1 L flask. Cells can then be cultured at about 36.0±0.5° C., 37.0±0.5° C., or 38.0±0.5° C. in a shaker incubator shaken at about 100, 150, 200, 225, 250, or about 300 rpm for about 10±1 h, 14±1 h, 16±1 h, or 18±1 h overnight or about 12 to 18 hours.

In one embodiment, the pH, temperature, dissolved oxygen concentration and osmolarity of the cell culture medium can depend on the type of host cell chosen. The pH, temperature, dissolved oxygen concentration (DO) and osmolarity can be selected for the growth and productivity of the cells. Depending on the protein and type of cell, the pH can be 6.6 to 7.6, in particular, at about 6.9, 7.0, 7.1 or 7.2, and the temperature can be about 30.0 to 39° C., in particular about 36.5° C., 36.8° C., 37.1° C., 37.4° C., 37.8° C., or 38.2º° C., and the osmolarity can be about 260 to 400 mOsm/kg.

Alternatively and/or additionally, silicon-based antifoams and defoamers or nonionic surfactants such as co-block polymers of ethylene oxide/propylene oxide monomers can be added to the medium during fermentation. The medium can be water.

In an embodiment, after overnight cell growth as described above, certain amounts of the culture can be transferred to a sterile container. The process is referred to as “seeding”. For example, about 100, 150, 200, 220, or 240 mL of the starting culture can be transferred to a sterile 250 mL seeding bottle and transfer into a fermenter for fed-batch fermentation. In another embodiment, another action can be performed at the same time as seeding. As defined above, the initial time of seeding can be referred to as S₀. In some embodiments, the host cell is polarized prior to the seeding step. Alternatively, or additionally, polarization can occur before fermentation.

In some embodiments, each fermenter can contain about 1-5 L, more particularly, about 1.0, 2.0, 2.5, 3.0, 3.2, 3.5, 3.7, 4.0, 4.3, 4.5 or 4.7 L of basic medium supplemented with about 1-5 g/L, more particularly, about 1.0, 2.0, 3.1, 3.5, 4.0, 4.5, or 5.0 g/L isoleucine, about 8-12 g/L basic glucose, in particular about 9.1, 9.5, 10.0, 10.5, or 11.0 g/L, about 10-25 μg/ml kanamycin, more particularly, about 12, 15, 20, or 25 μg/ml kanamycin, and about 15-30 μg/ml, more particularly, about 12, 15, 20, or 25 μg/ml, chloramphenicol. During fermentation, about 10-20 ml of the culture broth is typically taken at about 2.0 h interval to measure optical density at a light wavelength at 600 nm. The pH of the fed-batch fermentation is typically maintained and controlled at about pH 6.0-7.5, particularly at about 6.8, 6.9, 7.0, 7.1, 7.2, 7.3 or 7.4. All parameters in the present disclosure can be monitored and controlled via at least one automated adjustment controller, for example for adding NH₄OH to reach a final concentration of about 10-26%, in particular at about 11.0, 12.0, 12.5, 13.0, 13.5, 14.0, 14.5, 15.0, 15.5, 16.0, 17.0, 17.5, 18.0, 19.0, 20.0, 21.0, 22.0, 23.0, 24.0, 25.0, or 26.0%, or another buffer that can adjust the pH. The temperature is typically maintained at about 36.0 to 37° C., e.g., about 36.0, 36.5, and/or 37° C.

Each fermenter utilized in the methods and systems described herein can be any suitable size, for example, up to or larger than 200 L, e.g., 5 L, 10 L, 20 L, 30 L, 40 L, 50 L, 80 L, 100 L, 150 L, 200 L, or larger than 200 L. Each fermenter can have a minimum and maximum working volume depending on its capacity, which would be understood by a skilled artisan. In addition, as the fermentor size changes, either upwards or downwards, the concentrations of ingredients need not change, except for the carbon and nitrogen source that can be added, due to the fact that the carbon and nitrogen source additions are culture volume dependent. Nonlimiting examples are illustrated in Example 1-6, in which the carbon source added can be about a cumulative total about 800 to 1,500 g. When fermentor size increases, as denoted in Example 7, just for illustration, the carbon source added can amount to a cumulative total of about 6,000-9,000 g. Nonetheless, as the fermentor size changes upwards or downwards, changes in concentrations and conditions are also contemplated in the present disclosure,

Yet in another embodiment, typically, the performance of fed-batch fermentation can rely on process parameters such as substrate concentration, the substrate to inoculum ratio, retention time, and/or temperature. Moreover, the microbial community dynamics, their acclamations to changing operating conditions, and/or inhibitory conditions in the bioreactors can determine process stability. Among the several process parameters, the substrate to inoculum ratio (referred as “S: I” hereafter) can play a role in increasing fermentation/protein yield. The substrate fed (e.g., feeding strategy) to the bioreactors can alter the digestion process because of the difference in the quantity of organic components. Similarly, the inoculum, when supplied to the substrate, can provide functional stability. In an embodiment, the present disclosure's inoculum ratio is about 6.0 to 8.5%. Non-limiting examples include about 6.5 to 7%, 7.0 to 7.5%, 7.5 to 8%, or 8.0 to 8.5%; or about 6.25%, 6.3%, 6.4%, 6.5%, 6.67%, 6.7%, 6.8%, 6.95%, 7.0%, 7.24%, 7.3%, 7.33%, 7.48%, 7.5%, 7.6%, 7.76%, 7.8%, 7.9%, 8%, 8.14%, or 8.4%.

In another embodiment, the DO (dissolved oxygen) concentration of the culture medium can be influenced by the amount of air supplied to the bioreactor and the oxygen consumption of the growing cells. In the course of a bioprocess (e.g., fermentation) run, the DO typically will decrease, reflecting the oxygen consumption of the growing cells. If the carbon sources are depleted, the cells' metabolic activity and therefore their oxygen consumption can suddenly decrease, leading to a spike in the DO concentration. The DO spike therefore indicates substrate depletion and can be used to trigger automated culture feeding.

In an embodiment, the DO level of the fermentation can be maintained at about 25.0-45.0%, but not including 35.0%. For example, DO can be maintained at about 25.0%, 26.0%, 27.0%, 28.0%, 29.0%, 30.0%, 31.0%, 32.0%, 33.0%, 34.0%, 34.5%, 34.9%, 34.99%, 35.5%, 36.0%, 37.5%, 38.5%, 40.0%, 42.0%, 43.0%, or 45.0%. In another embodiment, the DO level of the fermentation can be maintained at about 25.0-45.0%, including 35.0%. For example, DO can be maintained at about 25.0%, 26.0%, 27.0%, 28.0%, 29.0%, 30.0%, 31.0%, 32.0%, 33.0%, 34.0%, 34.5%, 34.9%, 34.99%, 35.0%, 35.5%, 36.0%, 37.5%, 38.5%, 40.0%, 42.0%, 43.0%, or 45.0%. An automated method of maintaining air saturation can be used to automate feeding based on the DO spike by using a software controlled automated pump/sensor system. The apparatus can include one or more sensors to measure a fermentation's oxygen level, pH, redox measurement, and/or carbon source (e.g., glucose). The apparatus can also include a feed tubing for injection, gas supply for air/oxygen mixing, temperature control, pH control, and/or OD measurement apparatus. In an embodiment, the DO can be controlled by automated injection of oxygen to reach and/or adjust the desired level of DO. Alternatively, or additionally, the fermenter can be agitated and automatically controlled while monitoring DO. The speed of agitation can also affect DO. Non-limiting example include adjusting agitation rate at about 100 to 2,000 rpm.

In some cases, protein yield may not be optimal if one allows a fermentation batch at the end of the batch phases to exhibit a partial and/or full DO spike, which can lead to inconsistency of end product. Therefore, if the carbon sources are consumed to certain level, the present disclosure's method triggers an automated activation of feed pumps. In the present disclosure, one or more feeds can be added depending on fermentation conditions. For example, about 1, 2, 3, 4, 5, 6, 7, 8, 9, or 10 feeds with a carbon source feeding solution, typically comprising one or more carbon sources (e.g., glucose), can be injected into a fermenter during a fermentation run. This is referred to herein as carbon source feeding strategy.

In one embodiment, DO can gradually increase as fermentation progresses as the fermentation gradually increases the agitation rate from about 300 to 1,000 rpm (e.g., about 300, 400, 500, 600, 700, 800, 900 to about 1,000 rpm), which indicates supplying pure oxygen is needed.

In an embodiment of the present disclosure, at least one or more carbon source feeding strategy can be performed. Each carbon source feeding could be trigger by at least one of two sets of conditions: (i) when agitation exceeds about 300 to 1,000 rpm (particularly exceeding about 320, 350, 400, 450, 500, 600, 650, 700, 750, 800, 850, 900, 950 or 1,000 rpm) and DO exceeds about 25.0 to 45.0% (particularly exceeding about 35.0, 37.0, 38.0, 40.0, 42.5 or 45.0%); (ii) when agitation reaches above about 400 to 700 rpm (particularly about 400 to 500, 500 to 600, 600 to 700, 400, 450, 500, 550, 575, 600, 625 rpm or more), and pH rises above about 7.0 to 7.4 (particularly about 7.0 to 7.2, 7.2 to 7.4, 7.0, 7.2, 7.25, 7.30, or 7.4). In some embodiments, a carbon source feeding solution can contain about 600 to 900 g/L of D-(+)-Glucose Anhydrous and MgSO₄, particularly about 750, 800, or 850 g/L of D-(+)-Glucose Anhydrous, and about 20 to 600 g/L MgSO₄, particularly about 20, 80, 100, 200, 300, 400, 500, or 600 g/L MgSO₄.

In some embodiments, a cumulative total of about 800 to 1,500 g (e.g., about 850 to 950, 950 to 1,000, 1,000 to 1,100, 1,100 to 1,150, 1,150 to 1,200, 1,200 to 1,300, 1,300 to 1,400, 1,400 to 1,500, 850, 950, 1,000, 1,100, 1,200, 1,300, 1,400, or 1,500 g) of glucose can be fed to a fermenter having a 5 L capacity by the end of a fed-batch fermentation run. In another embodiment, a cumulative total of about 6,000 to 9,000 g (e.g., about 6,000 to 6,500, 6,5000 to 7,000, 7,000 to 7,500, 7,500 to 8,000, 8,000 to 8,500, 8,500 to 9,000, 6,000, 6,500, 7,000, 7,500, 8,000, 85,000, or 9,000 g) of glucose can be fed to a fermenter having a 40 L capacity by the end of a fed-batch fermentation run.

In another embodiment, about 30, 35, 40, 50, 55, 60 or 65 ml/L of Antifoam Y-30 emulsion can be added to prevent from and/or decrease over-foaming during the fed-batch fermentation. Alternatively, or additionally, about 80.0 to 100.0 grams of antifoam can be added at about I₂, I₄ I₆, and/or about 1.0 hr and/or 2.0 hr post IPTG addition.

In another embodiment, another feeding strategy called nitrogen source feeding can be utilized. The nitrogen source can be a yeast extract, peptone, soytone, urea, tryptone, or any combinations thereof. In an embodiment, the nitrogen source feeding strategy can include two or more feedings (e.g., 1, 2, 3, or 4) to a fermenter instead of known, typical single feeding. A nonlimiting example includes feeding a first nitrogen source at about I₀, I₁, or I₂ and a second nitrogen source at about at S₅, S₆, S₇, S₈, S₉, or S₁₀. Alternatively, or additionally, a feeding can be introduced when either one of the two sets of conditions below occurs: (1) agitation reaches above about 500-600 rpm, such as about 550 rpm, and DO level reaches above about 35-50%, such as about 40 or 45%; or (2) agitation reaches above about 500-600 rpm, such as about 550 or 600 rpm and pH reaches above about 7.1-7.3, such as about 7.2, 7.25 or 7.3. In one embodiment, a feeding is introduced when both sets of conditions (1) and (2) occur.

In an embodiment, the nitrogen source is a yeast extract (Y.E.) solution. A nonlimiting example includes adding at about I₀, I₁, or I₂ about 90 to 120 mL (e.g., 90 to 100, 100 to 110, 110 to 120, 90, 91, 96, 100, 101, 106, 110, 114, or 117 mL) of a Y.E solution. A second Y.E. solution can be added at about S₅, S₆, S₇, S₈, S₉, or S₁₀ at about 45 to 60 mL (e.g., 45 to 50, 50 to 55, 55 to 60, 50, 52, 54, 55, 56, 57, 58, 59, 60, 62, 64, 66, or 68 mL) of the Y.E solution. Alternatively, or additionally, a feeding can be introduced when either one of the two sets of conditions below occurs: (1) agitation reaches above about 500-600 rpm, such as about 550 rpm, and DO level reaches above about 35-50%, such as about 40 or 45%; or (2) agitation reaches above about 500-600 rpm, such as about 550 or 600 rpm and pH reaches above about 7.1-7.3, such as about 7.2, 7.25 or 7.3. In one embodiment, a feeding is introduced when both sets of conditions (1) and (2) occur.

In an embodiment, the centrifugation of any steps in the present disclosure used can be a continuously flow centrifuge. Alternatively, or additionally, the centrifugation can be done in a batch matter.

To one ordinary skilled in the art, potassium is not typically used in the prior art as part of the ingredient for Y.E. feeding. In one embodiment of the present disclosure, the Y.E. solution has a concentration about 320 to 560 g/L, particularly at about 385.0, 425.2, 428.6, 429.3, 450.7, 500.0, or 520.0 g/L. Unlike some methods, the present method can add potassium stock solution in the form of K₂HPO₄, at about 410-440 g/L, particularly about 415.4, 420, 421.5, 422.3, 425, 432.5, or 440 g/L for a final concentration reaching about 2.0-3.0 M; and/or KH₂PO₄ at about 190-230 g/L, particularity at about 192.3, 200.2, 205.7, 211.1, 225 g/L to reach a final concentration about 1.0 to 2.0 M. K⁺ solution can be added with feeding yeast extract solution so that the final potassium concentration of K+ is about 35 to 40 mM, particularly about 36, 37, 38 39, or 40 mM. In an embodiment, a Y.E. solution can be added at I₀ and one or more S₆, S₇, and S₈, or I₁ and S₅ and/or S₆, or I₀ and S₉ and/or S₁₀, with about 50 to 100 mL, particularly 50, 58, 65, 73, or 95 mL of a Y.E solution.

During fermentation, the expression of protein can be induced by addition of Isopropyl-beta-D-1-thiogalactopyranoside (IPTG) derived from a non-animal source, into a fermenter to achieve a final concentration about 0.5-2.5 mM, more particularly, about 0.5, 0.6, 0.7, 0.8, 0.9, 1.0, 1.2, 1.3, 1.4, 1.5, 2, 2.1, or 2.2 mM. In one embodiment, about 3 to 5 mL of about 0.70 to 0.85 mM of IPTG is added. In an embodiment, the initial induction can occur, and a second feeding yeast extract solution occurs when the OD₆₀₀ of the fed-batch culture reaches a value of about 40 to 80. For example, the induction chemical can be introduced to induce protein expression in the host cell when OD₆₀₀ value of the culture in the fermenter is at about 40 to 50, 50 to 60, 60 to 70, 70 to 80, 55 to 60, 60 to 65, 65 to 70, 70 to 75, 75 to 80, 40, 45, 49, 52, 55, 60, 66, 67, 68, 69, 70, 71, 72, 75, 78, or 80. After initial induction (I₀), the optical density of the fed-batch culture can be taken about every hour and recorded. Additionally, another feeding medium containing about 100-600 g/L of Y.E can be added, either separately, or in conjunction with IPTG, in particular, about 100, 117, 180, 200, 250, 280, 300, 330, 400, 420, 428, 430, 450, 453, 460, 500, 534, 550 g/L of Y.E.

In an embodiment, host cells expressing the protein of interest can be collected (e.g., harvested) at about 4-8 hours after IPTG induction (e.g., I₄ to I₈, I₄, I₅, I₆, I₇, or I₈), or for example, after about 5.0, 6.0, or 7.0 hours (15, 16, or I₇).

In an embodiment, some parameters and steps can be added or removed to increase yield. For example, certain step such as batch centrifugation step or addition of ammonium sulfate precipitation (ASP) buffer during purification can be removed. Other embodiments include adding or optimizing centrifugation of fermentation and homogenization, such as establishing centrifugation procedures in the present disclosure.

In another embodiment, the cell broth post fermentation can be collected and centrifuged at about 6,000-7,000 rpm, or about 7,000-8,000 rpm, such as at about 6,000, 6,500, 7,000, 7,500, or 8,000 rpm for about 10-30 minutes such as about 10, 15, 20, or 30 min at about 2-8° C. Supernatant can be discarded and the weight of cell pellet, namely the wet cell weight (WCW), can be recorded.

In some instances, harvested cell pellets can then be resuspended in about 10-50 mM TEN buffer (with pH=7.0±0.5) at about 7,000-12,000 rpm for about 20-50 seconds, and repeat for about 1 to 20 times, particularly, 8, 9, 10, 11 or 12 times. Suspension can be performed with stirring using a stainless-steel bar in a ratio of about 0.5 to 2.0 g, in particular, about 1.0, 1.3, 1.5 or 2.0 g wet cell pellet to about 3.0-6.0 mL TEN buffer. Following resuspension, the cell pellet/TEN buffer mixture can be homogenized more than once at about 800-1,200 bar, for example, at 800, 850, 900, 952, 1,000, 1052, 1,100, or 1,150 bar with a homogenizer. The cell mixture can then be centrifuged by continuous-flow centrifuge at about 10,000-30,000 rpm, such as about 10,000, 15,000, 25,000, 26,370 or 23,800 rpm. Alternatively, or additionally, a buffer feeding can be simultaneously performed at a rate about 35-45 ml/min, such as about 38, 40, or 43 ml/min, in a cooling coil and subsequently holding at about 20,000-30,000 rpm, such as at about 26,370, 25,000 or about 23,800 rpm for about 10-20 min.

Subsequently, in an embodiment, the cell pellets can be washed with 20 mM TEN buffer. After washing, the cell pellets can be fed to a centrifuge at a feeding rate about 10-40 ml/min, in particular, about 10, 20, 30, or 40 ml/min with centrifugation speed at about 10,000-30,000 rpm, for example, at about 20,000, 25,000, or 30,000 rpm for about 30-50 minutes, such as about 33, 42, or 50 minutes in the cooling coil and subsequently holding at about 25,000 rpm for about 15 min

In an embodiment, supernatants can then be discarded after centrifugation. The cell pellets can be mixed with about 3.0-5.0 M GnHCl (pH=˜7.0±0.5), in particular about 3.2, 4.0, or 4.5 M GnHCI for about 30-50 minutes, in particular, about 30, 35, 42, 47, or 50 minutes, to remove impurities.

In another embodiment, for each about 2.0-2.5 g wet cell pellet, about 0.5-3.0 mL of about 3.0-5.0 M GnHCI, in particular about 3.0, 4.0, or 4.5 M, can be added. After mixing using a stainless-steel bar, the solution can be transferred into a container with the quantity recorded. Alternatively, or additionally, the solution can be fed to a centrifuge at a feeding rate at about 25-38 ml/min with about 20,000-25,000 rpm for about 15-20 minutes in the cooling coil and subsequently holding at about 20,000-30,000 rpm, more particularly at about 22,000, 25,000, 28,000 rpm for about 25-35 min, and inclusion body can be collected and weighted.

In a non-limiting example, the above inclusion body can then be mixed with about 4.0-8.0 M of GdnHCl (pH=7.0±0.5), in particular about 6.0, 6.5, 6.8, 7.0, or 8.0 M GdnHCl, to solubilize the inclusion bodies. For each about 2.5 g wet cell pellet, about 1 ml of about 4.0-8.0 M GnHCI can be added and mixed. After mixing, about 1-3 M of Dithiothreitol (DTT), in particular about 1.0, 1.3, 1.5 or 2.0 M of DTT can be added to make a final DTT concentration of about 4-8 mM, in particular, about 4.0, 4.3, 5.0, 6.6 or 8.0 mM. The mixture can then be stirred at a stirring speed of about 100-500 rpm, particularly, about 135, 200, 366, 400, or 450 rpm at room temperature for about 1.0-3.0 hr, in particular, about 1.0, 1.5, or 2.0 hours. Following stirring, the solution can be transferred into 500 ml centrifuge tubes and centrifuged at about 10,000 to 30,000 rpm, in particular, about 10,000 or 20,000 rpm for about 40 minutes at about 20° C. The supernatants containing crude protein (e.g., crude protein extract) can be pooled and collected in a labeled plastic bottle. The protein can be analyzed using BCA (Bicinchoninic acid) protein assay.

Refolding of protein can be done using methods known in the art, for example, see U.S. Pat. No. 8,106,160 B2, the content of which is incorporated herein by reference in its entirety. Optionally, the crude protein in the crude protein extract can be separated by Q-Sepharose column chromatography.

The total, final fermentation time (e.g., when host cells in a fermenter are removed or harvested) can be about 10, 11, 12, 13, 14, 15, 16, 17, 18, 19, 24, 48, 72, 80 hours or more depending on fermentation conditions, type of protein, and desired yield and/or quality need.

Yet in an embodiment, the fed-batch fermentation process can run for a suitable amount of time. For example, the fermentation process can run for an amount of time in the range of about 0.5 to 72 hours, about 1.0 to 40 hours, about 2.0 to 10 hours, or greater than about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 80, 92 hours or more. The amount of time that fermentation is run can be controlled and can depend on the size of the fermenter.

In an embodiment, crude protein can be purified by Q Sepharose Fast Flow column to separate cellular debris and protein. see example 2 and/or 3 in U.S. Pat. No. 8,106,160 B2, the content of which is incorporated in reference in its entirety.

In some embodiments, proteins produced using the instant disclosure's method can additionally, and/or alternatively, undergo ion exchange chromatography for the purpose of purification. The ion exchange chromatography is typically an anion exchange chromatography. Anion exchange chromatography uses a positively charged ion exchange resin with an affinity for molecules having net negative surface charges. Anion exchange chromatography can be used both for preparative and analytical purposes and can separate a large range of molecules, from amino acids and nucleotides to large proteins.

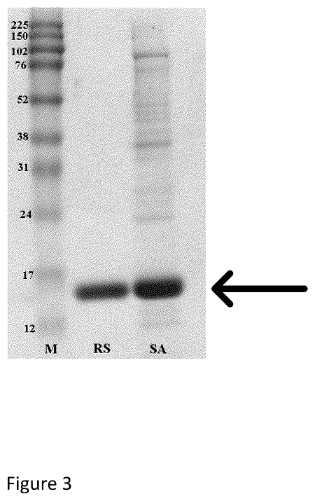

In an embodiment, the purified protein can then also be determined by reducing SDS-PAGE using 4-12% NuPAGE slab gels. FIG. 3 shows an example of SDS-PAGE gel of protein production with various induction time. The marked band shows the end product.

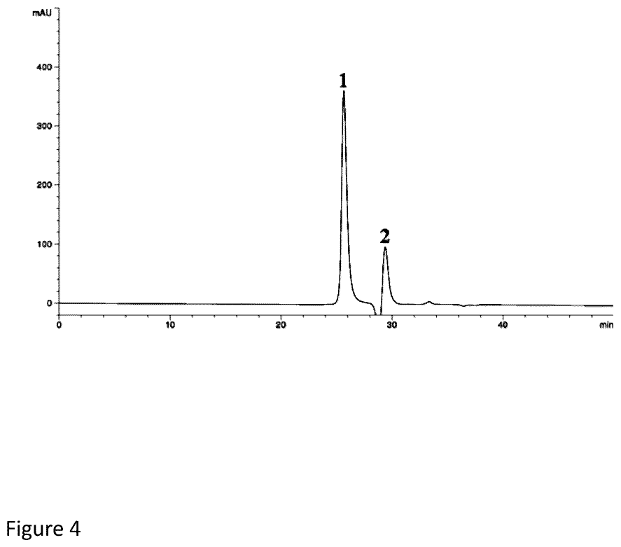

In another embodiment, the protein can alternatively and/or additionally be subjected to C₁₈ RP-HPLC and SEC-HPLC for quality assessment. An example can be seen in FIG. 4. Peak 2 as labelled can be solvent peak.

In another aspect of the disclosure, a method for manufacturing biopharmaceuticals is provided, which includes, but not limited to: providing at least one equipment configured for inputting cells; providing at least one equipment configured for inoculum expansion; providing at least one equipment configured for production stage perfusion; providing at least one equipment configured for volume exchange; providing at least one equipment configured for continuous purification; providing at least one equipment configured for virus reduction filtration; providing at least one equipment configured for ultrafiltration/diafiltration; providing at least one equipment configured for automated bulk fill; providing at least one connector between the at least one equipment configured for inputting cells and the at least one equipment configured for inoculum expansion; providing at least one connector between the at least one equipment configured for inoculum expansion and at least one equipment configured for production stage perfusion; providing at least one connector between the at least one equipment configured for production stage perfusion and the at least one equipment configured for volume exchange; providing at least one connector between the at least one equipment configured for volume exchange and the at least one equipment configured for continuous purification; providing at least one connector between the at least one equipment configured for continuous purification and the at least one equipment configured for virus reduction filtration; providing at least one connector between the at least one equipment configured for virus reduction filtration; providing at least one connector between the at least one equipment configured for ultrafiltration and diafiltration and the at least one equipment configured for automated bulk fill.

In another aspect of the disclosure, a manufacturing system for biopharmaceuticals can at least include, but not limited to: at least one equipment configured for inputting cells; at least one equipment configured for inoculum expansion; at least one production stage bioreactor, at least one equipment configured for primary recovery; at least one equipment configured for volume exchange; at least one equipment configured for continuous purification; at least one equipment for automated feeding during fermentation; at least one equipment configured for virus reduction filtration; and at least one equipment configured for automated bulk fill; and wherein the equipment are connected, and wherein the at least one equipment is connected through at least one piece of tubing and at least one connector such that the contents can pass from one piece of equipment to another via tubing.

In an embodiment, yield of the protein of interest after the fed-batch fermentation process described herein can be determined. In some embodiments, a crude protein extract obtained as described herein can be subjected to an ionic exchange column to remove large impurities. The protein concentration can be analyzed using BCA protein assay or other methods known in the art.

In an embodiment, utilizing the present disclosure's methods or systems can increase the yield of protein of interest in crude protein after fermentation, by at least about 5%, at least 10%, at least 15%, at least 20%, at least 25%, at least 30%, at least 35%, at least 40%, at least 45%, at least 50%, at least about 51.7%, at least 55%, at least 60%, at least about 62.7%, at least 65%, at least 70%, at least about 73.7%, at least 75%, at least 80%, at least about 84.7%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 383%, at least 400%, at least 450%, at least 500%, at least 550%, at least 600%, at least 650%, at least 700%, at least 748%, at least 750%, at least 800%, at least 9 folds, at least 10 folds, at least 20 folds, or more, as compared to existing known method for producing crude protein from fermentation of such protein of interest. In some embodiments, in the case of a 40 L fermenter, the yield of the present method can be increased by at least about 100% to 200%, 100%, 105%, 110%, 115%, 120%, 125%, 130%, 135%, 140%, 145%, 150%, 155%, 160%, 165%, 170%, 175%, 180%, 190%, or 200%, as compared to 8 times the yield of a fermenter having about a 5 L capacity in the existing known fermentation method.

In one embodiment, utilizing the present disclosure's methods or systems can increase the yield of protein of interest in crude protein after fermentation to about 5 to 10 g or more (e.g., about 5, 5.5, 6, 6.5, 7, 7.5, 8, 8.5, 9, 9.5, 10 g or more) of the protein of interest in a fermenter having about a 5 L capacity. In another embodiment, utilizing the present disclosure's methods or systems can increase the yield of protein of interest in crude protein after fermentation to about 20 to 30 g or more (e.g., about 20, 21, 22, 23, 24, 25, 26, 27, 28, 29, 30 g or more) of the protein of interest in a fermenter having about a 40 L capacity.

Yet in an embodiment, utilizing the present disclosure's methods or systems increases the total protein yield of protein of such interest's produced after fermentation and inclusion body processing, wherein the end protein yield can be increased by at least about 5%, at least 10%, at least 15%, at least 20%, at least 25%, at least 30%, at least 35%, at least 40%, at least 45%, at least 50%, at least about 51.7%, at least 55%, at least 60%, at least about 62.7%, at least 65%, at least 70%, at least about 73.7%, at least 75%, at least 80%, at least about 84.7%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 383%, at least 400%, at least 450%, at least 500%, at least 550%, at least 600%, at least 650%, at least 700%, at least 748%, at least 750%, at least 800%, at least 9 folds, at least 10 folds, at least 20 folds, or more, as compared to existing known method for producing crude protein from fermentation of such protein of interest. In some embodiments, in the case of a 40 L fermenter, the yield of the present method can be increased by at least about 100% to 200%, 100%, 105%, 110%, 115%, 120%, 125%, 130%, 135%, 140%, 145%, 150%, 155%, 160%, 165%, 170%, 175%, 180%, 190%, or 200%, as compared to 8 times the yield of a fermenter having about a 5 L capacity in the existing known fermentation method.

Yet in an embodiment, utilizing the present disclosure's methods or systems increases the total protein yield of final purified protein of interest produced after fermentation, inclusion body processing, and purification, wherein the end protein yield can be increased by at least about 5%, at least 10%, at least 15%, at least 20%, at least 25%, at least 30%, at least 35%, at least 40%, at least 45%, at least 50%, at least about 51.7%, at least 55%, at least 60%, at least about 62.7%, at least 65%, at least 70%, at least about 73.7%, at least 75%, at least 80%, at least about 84.7%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 383%, at least 400%, at least 450%, at least 500%, at least 550%, at least 600%, at least 650%, at least 700%, at least 748%, at least 750%, at least 800%, at least 9 folds, at least 10 folds, at least 20 folds, or more, as compared to existing known method for producing crude protein from fermentation of such protein of interest. In some embodiments, in the case of a 40 L fermenter, the yield of the present method can be increased by at least about 100% to 200%, 100%, 105%, 110%, 115%, 120%, 125%, 130%, 135%, 140%, 145%, 150%, 155%, 160%, 165%, 170%, 175%, 180%, 190%, or 200%, as compared to 8 times the yield of a fermenter having about a 5 L capacity in the existing known fermentation method.

In an embodiment, utilizing the present disclosure's methods or systems can increase the yield of crude protein of interest, such as all types of interferon (Type I, Type II, a, b, or alpha, beta . . . etc.). The crude interferon protein produced after fermentation can be increased by at least about 5%, at least 10%, at least 15%, at least 20%, at least 25%, at least 30%, at least 35%, at least 40%, at least 45%, at least 50%, at least about 51.7%, at least 55%, at least 60%, at least about 62.7%, at least 65%, at least 70%, at least about 73.7%, at least 75%, at least 80%, at least about 84.7%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 383%, at least 400%, at least 450%, at least 500%, at least 550%, at least 600%, at least 650%, at least 700%, at least 748%, at least 750%, at least 800%, at least 9 folds, at least 10 folds, at least 20 folds, or more, as compared to existing known method for producing crude interferon protein from fermentation of such protein of interest. In some embodiments, in the case of a 40 L fermenter, the yield of the present method can be increased by at least about 100% to 200%, 100%, 105%, 110%, 115%, 120%, 125%, 130%, 135%, 140%, 145%, 150%, 155%, 160%, 165%, 170%, 175%, 180%, 190%, or 200%, as compared to 8 times the yield from a fermenter having about a 5 L capacity in the existing known fermentation method.

In yet another embodiment, utilizing the present disclosure's methods or systems can increase the yield of crude protein of interest, such as all types of interferon (Type I, Type II, a, b, or alpha, beta . . . etc.). The crude interferon protein produced after fermentation can be increased to about 5 to 10 g or more (e.g., about 5, 5.5, 6, 6.5, 7, 7.5, 8, 8.5, 9, 9.5, 10 g or more) of the protein of interest in a fermenter having about a 5 L capacity. In another embodiment, the crude interferon protein produced after fermentation can be increased to about 20 to 30 g or more (e.g., about 20, 21, 22, 23, 24, 25, 26, 27, 28, 29, 30 g or more) of the protein of interest in a fermenter having about a 40 L capacity.

Yet in an embodiment, utilizing the present disclosure's methods or systems increases the total purified protein yield for protein such as interferon (a, b, or alpha, beta . . . etc.) after fermentation, inclusion body processing, and purification steps, wherein the end interferon yield can be increased by at least about 5%, at least 10%, at least 15%, at least 20%, at least 25%, at least 30%, at least 35%, at least 40%, at least 45%, at least 50%, at least about 51.7%, at least 55%, at least 60%, at least about 62.7%, at least 65%, at least 70%, at least about 73.7%, at least 75%, at least 80%, at least about 84.7%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 383%, at least 400%, at least 450%, at least 500%, at least 550%, at least 600%, at least 650%, at least 700%, at least 748%, at least 750%, at least 800%, at least 9 folds, at least 10 folds, at least 20 folds, or more, as compared to existing known method for producing crude protein from fermentation of such interferons. In some embodiments, in the case of a 40 L fermenter, the yield of the present method can be increased by at least about 100% to 200%, 100%, 105%, 110%, 115%, 120%, 125%, 130%, 135%, 140%, 145%, 150%, 155%, 160%, 165%, 170%, 175%, 180%, 190%, or 200%, as compared to 8 times the yield of a fermenter having about a 5 L capacity in the existing known fermentation method.

In an embodiment, utilizing the present disclosure's methods or systems increases the yield of protein of interest, such as proline interferon alpha-2b. The crude proline interferon alpha-2b protein produced after fermentation can be increased by at least about 5%, at least 10%, at least 15%, at least 20%, at least 25%, at least 30%, at least 35%, at least 40%, at least 45%, at least 50%, at least about 51.7%, at least 55%, at least 60%, at least about 62.7%, at least 65%, at least 70%, at least about 73.7%, at least 75%, at least 80%, at least about 84.7%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 383%, at least 400%, at least 450%, at least 500%, at least 550%, at least 600%, at least 650%, at least 700%, at least 748%, at least 750%, at least 800%, at least 9 folds, at least 10 folds, at least 20 folds, or more, as compared to existing known method for producing crude proline interferon alpha-2b from fermentation of such protein of interest. In some embodiments, in the case of a 40 L fermenter, the yield of the present method can be increased by at least about 100% to 200%, 100%, 105%, 110%, 115%, 120%, 125%, 130%, 135%, 140%, 145%, 150%, 155%, 160%, 165%, 170%, 175%, 180%, 190%, or 200%, as compared to 8 times the yield from a fermenter having about a 5 L capacity in the existing known fermentation method.

In another embodiment, utilizing the present disclosure's methods or systems increases the yield of protein of interest, such as proline interferon alpha-2b. The crude proline interferon alpha-2b protein produced after fermentation can be increased to about 5 to 10 g or more (e.g., about 5, 5.5, 6, 6.5, 7, 7.5, 8, 8.5, 9, 9.5, 10 g or more) of the protein of interest in a fermenter having about a 5 L capacity or to about 20 to 30 g or more (e.g., about 20, 21, 22, 23, 24, 25, 26, 27, 28, 29, 30 g or more) of the protein of interest in a fermenter having about a 40 L capacity.

Yet in an embodiment, utilizing the present disclosure's methods or systems increases the total protein yield of protein such as proline interferon alpha-2b after fermentation, inclusion body processing, and purification, wherein the end proline interferon alpha-2b yield increased by at least about 5%, at least 10%, at least 15%, at least 20%, at least 25%, at least 30%, at least 35%, at least 40%, at least 45%, at least 50%, at least about 51.7%, at least 55%, at least 60%, at least about 62.7%, at least 65%, at least 70%, at least about 73.7%, at least 75%, at least 80%, at least about 84.7%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 383%, at least 400%, at least 450%, at least 500%, at least 550%, at least 600%, at least 650%, at least 700%, at least 748%, at least 750%, at least 800%, at least 9 folds, at least 10 folds, at least 20 folds, or more, as compared to existing known method for producing crude or purified protein of proline interferon alpha-2b from fermentation. In some embodiments, in the case of a 40 L fermenter, the yield of the present method can be increased by at least about 100% to 200%, 100%, 105%, 110%, 115%, 120%, 125%, 130%, 135%, 140%, 145%, 150%, 155%, 160%, 165%, 170%, 175%, 180%, 190%, or 200%, as compared to 8 times the yield from a fermenter having about a 5 L capacity in the existing known fermentation method.

In an embodiment, utilizing the present disclosure's methods or systems can increase the yield of crude protein GCSF. The crude GCSF protein produced after fermentation can be increased by at least about 5%, at least 10%, at least 15%, at least 20%, at least 25%, at least 30%, at least 35%, at least 40%, at least 45%, at least 50%, at least about 51.7%, at least 55%, at least 60%, at least about 62.7%, at least 65%, at least 70%, at least about 73.7%, at least 75%, at least 80%, at least about 84.7%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 383%, at least 400%, at least 450%, at least 500%, at least 550%, at least 600%, at least 650%, at least 700%, at least 748%, at least 750%, at least 800%, at least 9 folds, at least 10 folds, at least 20 folds, or more, as compared to existing known method for producing crude GCSF from fermentation of such protein of interest. In some embodiments, in the case of a 40 L fermenter, the yield of the present method can be increased by at least about 100% to 200%, 100%, 105%, 110%, 115%, 120%, 125%, 130%, 135%, 140%, 145%, 150%, 155%, 160%, 165%, 170%, 175%, 180%, 190%, or 200%, as compared to 8 times the yield of a fermenter having about a 5 L capacity in the existing known fermentation method.

Yet in an embodiment, utilizing the present disclosure's methods increases the total protein yield of purified GCSF after fermentation, inclusion body processing, and purification, wherein the end GCSF yield is increased by at least about 5%, at least 10%, at least 15%, at least 20%, at least 25%, at least 30%, at least 35%, at least 40%, at least 45%, at least 50%, at least about 51.7%, at least 55%, at least 60%, at least 62.7%, at least 65%, at least 70%, at least about 73.7%, at least 75%, at least 80%, at least about 84.7%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 383%, at least 400%, at least 450%, at least 500%, at least 550%, at least 600%, at least 650%, at least 700%, at least 748%, at least 750%, at least 800%, at least 9 folds, at least 10 folds, at least 20 folds, or more, as compared to existing known method for producing crude protein and/or purified GCSF from fermentation. In some embodiments, in the case of a 40 L fermenter, the yield of the present method can be increased by at least about 100% to 200%, 100%, 105%, 110%, 115%, 120%, 125%, 130%, 135%, 140%, 145%, 150%, 155%, 160%, 165%, 170%, 175%, 180%, 190%, or 200%, as compared to 8 times the yield of a fermenter having about a 5 L capacity in the existing known fermentation method.

In an embodiment, utilizing the present disclosure's methods can increase the yield of crude protein anti-PD-1 antibody. The crude anti-PD-1 antibody protein produced after fermentation can be increased by at least about 5%, at least 10%, at least 15%, at least 20%, at least 25%, at least 30%, at least 35%, at least 40%, at least 45%, at least 50%, at least about 51.7%, at least 55%, at least 60%, at least about 62.7%, at least 65%, at least 70%, at least about 73.7%, at least 75%, at least 80%, at least about 84.7%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 383%, at least 400%, at least 450%, at least 500%, at least 550%, at least 600%, at least 650%, at least 700%, at least 748%, at least 750%, at least 800%, at least 9 folds, at least 10 folds, at least 20 folds, or more, as compared to existing known method for producing crude anti-PD-1 antibody from fermentation of such protein of interest. In some embodiments, in the case of a 40 L fermenter, the yield of the present method can be increased by at least about 100% to 200%, 100%, 105%, 110%, 115%, 120%, 125%, 130%, 135%, 140%, 145%, 150%, 155%, 160%, 165%, 170%, 175%, 180%, 190%, or 200%, as compared to 8 times the yield of a fermenter having about a 5 L capacity in the existing known fermentation method.

Yet in an embodiment, utilizing the present disclosure's methods increases the total protein yield of purified anti-PD-1 antibody after fermentation, inclusion body processing, and purification, wherein the end anti-PD-1 antibody yield is increased by at least about 5%, at least 10%, at least 15%, at least 20%, at least 25%, at least 30%, at least 35%, at least 40%, at least 45%, at least 50%, at least about 51.7%, at least 55%, at least 60%, at least 62.7%, at least 65%, at least 70%, at least about 73.7%, at least 75%, at least 80%, at least about 84.7%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 350%, at least 383%, at least 400%, at least 450%, at least 500%, at least 550%, at least 600%, at least 650%, at least 700%, at least 748%, at least 750%, at least 800%, at least 9 folds, at least 10 folds, at least 20 folds, or more, as compared to existing known method for producing crude protein and/or purified anti-PD-1 antibody from fermentation. In some embodiments, in the case of a 40 L fermenter, the yield of the present method can be increased by at least about 100% to 200%, 100%, 105%, 110%, 115%, 120%, 125%, 130%, 135%, 140%, 145%, 150%, 155%, 160%, 165%, 170%, 175%, 180%, 190%, or 200%, as compared to 8 times the yield of a fermenter having about a 5 L capacity in the existing known fermentation method.

### Non-Limiting Mode of Operation

In an embodiment, the monitoring of agitation speed, pH, and/or dissolved oxygen can be performed continuously throughout the fermentation process with carbon and/or nitrogen source added as need. Alternatively, or additionally, the monitoring can be done at a fixed time intervals. Non-limiting examples include about every 1, 1.5, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22.5, and/or 23 hours. Alternatively, or additionally, the fixed time intervals can be every 30 minutes up to 80 hours or longer, depending on fermentation scale. In other embodiments, the additional of total cumulative carbon and/or nitrogen source can depend on whether a continuous or batch monitoring of agitation speed, pH, and/or dissolved oxygen is performed.

### Surprising and Unexpected Results

It is known in the art that scaling up a protein production in a bioprocess is challenging and unpredictable. For example, scaling up eight-fold such as from a 5 L to a 40 L fermenter would not result in a corresponding eight-fold increase in yield. Indeed, the typical result is a decrease in yield relative to the scale-up; it is known that increase in volume does not provide a linear theoretical increase. In addition, as the degree of scaling up increases, the yield not only does not exhibit a linear increase, it exhibits a curvature decrease as the bioprocess increases in size.

Therefore, the present disclosure demonstrates more than one unexpected result. For example, adding more steps would typically decrease stepwise yield, but the additional steps in the methods described herein present the opposite effect of leading to yield increase. In addition, the present disclosure demonstrates that the conditions disclosed herein, when used in larger scale fermentation (e.g., 40 L fermenter), does not exhibit the typical decrease in yield in a curvature manner as volume increases. In fact, the present disclosure shows that, the yield of the fermentation method described herein not only can increase pass the linear theoretical yield (i.e., multiplying the yield of a smaller scale fermentation by the fold of fermenter volume increase) of the known method but substantially exceeds such theoretical yield (see, e.g., the data presented in Example 7 below). Therefore, the present disclosure demonstrates unexpected results teaching away from what is known in the art and surprising superiority of the improved methods described herein.

### EXAMPLES

### Example 1: Method of Making Pro-hIFN-α2b from Known Method

U.S. Pat. No. 8,106,160 B2 discloses a known method of fermenting cells to make a protein named Pro-hIFN alpha 2b. In this Example, the previously disclosed method was followed by carrying out the steps as described. More particularly, the follows steps were carried out.

A first nucleic acid encoding hIFN-α2b was amplified by PCR using human genomic DNA as a template. The PCR primers were designed based on the flanking sequences of the coding region of the hIFN-α2b gene. The PCR product thus obtained was cloned into a vector and then subcloned into pET-24a, a protein expression vector.

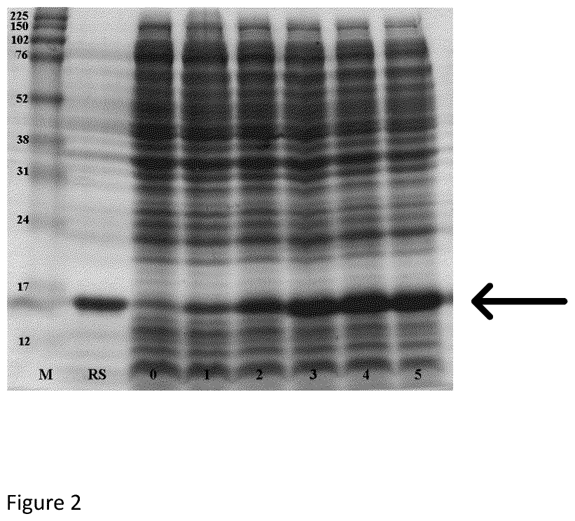

A second nucleic acid encoding Pro-hIFN-α2b was obtained by modifying the first nucleic acid described above via PCR amplification using primers designed according to the nucleic acid sequence shown in FIG. 2 of U.S. Pat. No. 8,106,160 B2. Briefly, two additional codons, encoding Met and Pro were added to the 5′ of codon TGT that encodes Cys₁ of mature hIFN-α2b. This nucleic acid was also cloned into expression vector pET-24a.

pET-24a vectors, carrying the hIFN-α2b and Pro-hIFN-α2b genes, were then transformed into E. coli BLR-CodonPlus (DE3)-RIL strain. E. coli clones expressing high levels of these two proteins were selected. As designed, the nascent protein expressed from the Pro-hIFN-α2b gene has a Met at its N-terminus (Met-Pro-Cys-). This Met residue was removed in E. coli via internal enzymatic digestion, resulting in a mature protein having an N-terminal Pro, which is linked to the Cys₁ of hIFN-α2b (Pro-Cys-).

The E. coli clone expressing either hIFN-α2b or Pro-hIFN-α2b was cultured in a 1,000 ml flask containing 250 ml SYN Broth medium (soytone, yeast extract, and NaCl) with kanamycin (50 μg/mL) and chloramphenicol (50 μg/mL) at 37° C., 200 rpm for 16 hours. 220 ml of the overnight culture were then transferred to a 5-liter jar fermenter containing 3 L define medium (10 g/L glucose, 0.7 g/L MgSO₄·7H₂O, 4 g/L (NH₄) 2HPO₄, 3 g/L KH₂PO₄, 6 g/L K₂HPO₄, 1.7 g/L citrate, 10 g/L yeast extract, 10 ml/L Trace Element, and g/L isoleucine) with kanamycin (25 μg/mL), chloramphenicol (25 μg/mL), 0.4% glycerol, and 0.5% (v/v) trace elements (10 g/L of FeSO₄·7H₂O, 2.25 g/L of ZnSO₄·7H₂O, 1 g/L of CuSO₄·5H₂O, 0.5 g/L of MnSO₄·H₂O, 0.3 g/L of H₃BO₃, 2 g/L of CaCl₂·2H₂O, 0.1 g/L of (NH₄)₆Mo₇O₂₄, 0.84 g/L EDTA, and 50 ml/L HCl).

The oxygen concentration in the medium was controlled at 30-40%, but not at 35% and its pH maintained at about 7.0 by adding about 25% ammonia water whenever necessary. A feeding solution containing about 700-800 g/L of glucose and about 15-20 g/L of MgSO₄·7H₂O was prepared. When the dissolved oxygen rose to a value greater than the set point, an appropriate volume of the feeding solution was added to increase the glucose concentration in the culture medium. The expression of the hIFN-α2b or the Pro-hIFN-α2b gene was induced by IPTG at a final concentration of about 0.5-0.85 mM, then addition of feeding material (yeast extract and trace element). E. coli cells expressing these proteins were collected after about 3-5 hours after IPTG induction.

The collected E. coli cells were resuspended in TEN buffer (50 mM Tris-HCl, pH 7.0; 1 mM EDTA, and 100 mM NaCl) in a ratio of about 1:10 (wet weight g/mL), disrupted by a homogenizer, and then centrifuged at 10,000 rpm for 20 min.

The pellet containing inclusion bodies (IBs) was washed twice with TEN buffer and centrifuged as described above, suspended in a ratio of about 1 ml solution: 2.5 g pellet wet weight g/mL of a 4 M guanidium HCl (GnHCl) aqueous solution, and then centrifuged at 20,000 rpm for 20 min. The IBs, containing recombinant hIFN-α2b, were then solubilized in about 50 mL of 6 M GnHCl with about 5 mM DTT, which was then stirred at room temperature for 1.5 hr followed by centrifugation at 20,000 rpm for 20 min at 25° C. The supernatant was collected. In this process, the recombinant hIFN-α2b or Pro-hIFN proteins were denatured.

13% SDS-PAGE analysis was performed using method known in the art. The data exhibited increasing concentration of protein as induction time increases (data not shown).

The above-described IBs were mixed with 1.5 L freshly prepared refolding buffer (about 100 mM Tris-HCl (pH 7.0), about 0.5 M L-Arginine, and about 2 mM EDTA). The reaction mixture thus formed was incubated for about 24-36 hr without stirring at room temperature to allow refolding of the recombinant hIFN-α2b and Pro-hIFN-α2b proteins. The refolded proteins were then dialyzed against about 20 mM Tris-HCl buffer, pH 7.0 and further purified by Q-Sepharose column chromatography described below.

A Q-Sepharose column was pre-equilibrated and washed with a 20 mM Tris-HCl buffer (pH 7.0). The refolded recombinant proteins were then loaded onto the equilibrated Q-Sepharose column and eluted with a 20 mM Tris-HCl buffer (pH 7.0) containing about 80 mM NaCl. Fractions containing Pro-hIFN-α2b were collected based on their absorbance at 280 nm. The concentrations of these proteins were determined by a protein assay kit using the Bradford method.

The conformation of the refolded recombinant proteins was determined using C₁₈ reverse phase HPLC (RP-HPLC), in which native-formed proteins and slow monomers were separated. The RP-HPLC analysis was conducted on a HPLC system equipped with an automated gradient controller, two buffer pumps, UV detector, and a recorder-integrator. A C₁₈ HPLC column (46 mm×250 mm, 5 μm particle size; 300 Å pore size) was equilibrated in 80% buffer A: 0.2% (v/v) TFA in 30:70 acetonitrile:water (HPLC grade). The proteins were eluted from the C₁₈ HPLC column using buffer B (0.2% TFA in 80:20 acetonitrile:water) at a flow rate of 1 ml/min according to the following gradient:

| Time | | |
|---|---|---|
| (min) | Eluent A | Eluent B |
| 0 | 72 | 28 |
| 1 | 72 | 28 |
| 5 | 67 | 33 |
| 20 | 63 | 37 |
| 30 | 57 | 43 |
| 40 | 40 | 60 |
| 42 | 40 | 60 |
| 50 | 72 | 28 |

Results obtained from the C₁₈ HPLC analysis indicate that a substantial amount of the refolded recombinant hIFN-α2b proteins are slow monomers. Differently, there is only little contamination of slow monomers in refolded recombinant Pro-hIFN-α2b proteins.

hIFN-α2b was further purified by Q-Sepharose column chromatography as described above. hIFN-α2b isoforms, including oligomers and slow monomers, contained in the purified proteins were removed according to the method described in U.S. Pat. No. 4,534,906. Briefly, the proteins were mixed with ASP buffer (about 3 M ammonium sulfate and about 1 M NaOAc) to the final concentration (about 0.9 M ammonium sulfate and about 20 mM NaOAc) at the pH value of about 4.5. The mixture was incubated at room temperature (e.g., 34 to 40° C.) for 20 minutes to effect formation of protein precipitates, which include slow monomers and oligomers. The precipitates were removed by centrifugation and the supernatant containing hIFN-α2b in native form was collected. The proteins samples before and after ammonium sulfate/sodium chloride treatment were analyzed in C18 reverse phase HPLC. hIFN-α2b proteins prepared by Q-Sepharose column purification include a certain number of isoforms. These isoforms have been removed after ammonium sulfate/sodium chloride treatment.

### Example 2: Amount of Protein Yield from Known Method

The crude protein ran through an ionic exchange column to remove impurities. Afterwards, some purified proteins were run against another SDS-PAGE performed using method known in the art to determine whether the protein produced matches the size of a standard.

Table 1 shows the result of above technique, where the left column shows the batch number, and the right column shows the protein yield in the parentheses, along with new batch number. In addition, as described in one embodiment above, two (2) or more batches of fermentation can be made and combine into a single batch in order to have sufficient protein to perform any additional chemical modifications desirable. Table 1 depicts the yield information for the protein using the known method, for example, for U.S. Pat. No. 8,106,160.

TABLE 1

| Batch | Protein |
|---|---|
| number | Yield |
| 7002 | 4.26 g |
| 7003 | |
| 7004 | 4.42 g |
| 7007 | |
| 7008 | 4.04 g |
| 7009 | |

Batch 7002 and 7003 were separately fermented each in 5 L volume, combined. Similarly, batch 7004 and 7007 were separately fermented and combined. Likewise for batches 7008 and 7009. The average yield for 6 batches of fermentation is about 2.12 g of the protein of interest per a 5 L fermenter.

### Example 3

### New Method of Making Pro-hIFN-α2b

Unlike Examples 1-2, this Example illustrates a novel fermentation method to improve protein yield, even when more steps were added, the result of which is surprising and unexcepted. These new steps are not routine optimization by one ordinary skilled in the art. Here, the particular protein chosen is also Proline N-terminally modified IFNa-2b, for a comparison of yield between Example 1 and 3.

In this example, a vial of recombinant E. coli BLR (DE3) to which expression vector for expressing Methionine-Proline N-terminally modified IFNa-2b gene has already been transfected by methods known in the art. The cell was taken from liquid nitrogen. After thawing and equilibration to room temperature, the E. coli cells were transferred into 250 ml of the SYN medium in a sterile 1 L flask. The medium was pre-mixed with about 50 g/ml kanamycin and about 50 μg/ml chloramphenicol. Cells were then cultured at 37° C. in a shaker incubator at about 100-200 rpm for about 10 to 20 hours of culture, in particular, about 16±1 h overnight culture.

After cell growth, about 200 to 230 ml, for example, about 205, 210 or 220 ml of the starting culture was separately transferred into sterile 250 ml seeding bottles in a laminar flow hood and then pumped into fermenters. Initially, each fermenter contained about 1-3 L of basic medium supplemented with about 1-3 g/L isoleucine, such as about 1.4, 2.0, or 2.6 g/L isoleucine, about 5-10 g/L basic glucose, about 25 μg/ml kanamycin, and about 25 μg/ml chloramphenicol and polarized at about 36-38° C., such as 36.5 or 37° C. for overnight. The culture was then fed into a 5 L fermenter and inoculated for about 3-5 minutes, which is considered as the zero-time-point for measuring cell growth. During fermentation, about 10-20 ml of the culture broth was individually taken at about 1.0-2.0 h interval and subjected to absorption spectrometer to measure optical density at 600 nm. The pH of the fed-batch fermentation was controlled at about pH 7.0-7.5, such as about pH 7.1, 7.2, or 7.5 by automated addition of NH₄OH. The dissolved oxygen (DO) level was controlled to be at less than about 35.0% (but not equal to 35.0%, for example, at 30.0%, occasionally it could cross 35.0% to reach a higher DO) of air saturation as the agitation rate gradually increased from about 300 to about 1,000 rpm. Pure oxygen was supplied when agitation speed reached its upper limit and more oxygen was required. Glucose solution was added using a program-controlled pump that was set to feed when (1) DO level exceeded (or spiked above) about 30-40% or above about 40%, and agitation rate exceeded about 350-450 rpm; and/or (2) when agitation rate exceeded about 500-650 rpm and pH exceeded about 7.0-7.2. About 30-50 ml/L of antifoam Y-30 emulsion was added to prevent over-foaming during the fed-batch fermentation. About 50-60 ml of feeding yeast extract solution was pumped into the fermenter after initial inoculated (seed) at about 5.0, 6.0, 7.0, or 8.0 hours (or S₅, S₆, S₇ or S₈) post seeding depending on OD₆₀₀ or overall fermentation conditions. The expression of protein was achieved by addition of about 3 to 5 ml of about 0.75, 1.0, or 1.5 M IPTG (also known as initial induction, or I₀) into the fermenter along with about 120 ml of feeding yeast extract solution when the OD₆₀₀ of the fed-batch culture reached a value of about 70-80, for example, about 71, 73, 75, 77, 79, or 80. The exact amount depends on OD₆₀₀ and overall fermentation conditions. After induction, the optical density of the fed-batch culture was taken every hour and recorded.

After about 4-8 hours of IPTG induction, (I₄ to I₈), the fed-batch culture was harvested. The cell broth was collected in about a 500 ml centrifugation bottle and centrifuged at about 7,000-9,000 rpm for about 10-20 minutes at 4° C. Supernatant was then discarded and the weight of cell pellet, namely the wet cell weight (WCW), was recorded. The expression level of protein was determined by reducing SDS-PAGE using 4-12% NuPAGE slab gels. The cell pellets were then transferred into a storage box.

The cell pellets obtained from the cell harvest step were resuspended completely in 20 mM TEN buffer (pH=7.0±0.5) with about 7,000 to about 12,000 rpm stirring using a stainless-steel bar in a ratio of about 1 g wet cell pellet to about 1.0, 3.0, or 5.0 ml TEN buffer. Following complete resuspension, the cell pellet/TEN buffer mixture was homogenized twice at about 800-1,200 bar, particularly at about 950, 1,000, 1,050, or 1,100 bar with a homogenizer. The cell mixture was then centrifuged by continuous-flow centrifugation at about 20,000-30,000 rpm with a feeding of materials needing to be centrifuged again at a rate about 30-50 ml/min in the cooling coil, and subsequently holding at about 20,000-30,000 rpm for about 10-30 min. The pellet was collected after centrifugation.

The cell pellets were washed with 20 mM TEN buffer (pH=7.0±0.5) at the ratio of about 0.8-1.2 g wet cell pellet to 1.0 to 3 ml TEN buffer. After washing, the cell pellets were fed to centrifuge at a feeding rate about 30-40 ml/min with centrifugation speed at about 25,000 rpm for about 40-45 minutes in the cooling coil and subsequently holding at about 20,000-25,000 rpm for about 10-15 min. Supernatants were discarded after continuous-flow centrifugation.

The cell pellets were then mixed with about 4-6 M GnHCI (pH=7.0±0.5) for about 30 minutes to remove unwanted proteins. For each about 2.0-2.5 g wet cell pellet, about 0.5-1.0 ml of about 4-6 M of GnHCI was added. After thorough mixing using a stainless-steel bar, the solution was transferred into 1 L beaker with the quantity recorded. The solution was fed to a centrifuge at a feeding rate about 20-30 ml/min with about 20,000-25,000 rpm for about 10-20 minutes in the cooling coil and subsequently holding at about 20,000-25,000 rpm for about 20-30 min. Supernatants were discarded after centrifugation and doubly washed inclusion bodies were collected.

The above washed inclusion bodies were then mixed with about 4.0-6.0 M GdnHCI to solubilize the inclusion bodies. For each about 2.0-2.5 g wet cell pellet, about 0.5-1.0 ml GnHCI was added. After complete mixing, the solution was transferred into a plastic bottle; and a designated amount of about 0.5 to 2.0 M Dithiothreitol (DTT) was then added to make a final DTT concentration of about 4.0-6.0 mM. The sample mixture was then stirred at a stirring speed of about 200 rpm at room temperature for about 1.0-3.0 hours. Following stirring, the solution was transferred back into the 500 ml centrifuge tubes. The supernatants containing protein were pooled and collected in labeled plastic bottle.

### Example 4

### Protein Assay

As shown in FIG. 2, post IPTG induction cell lysate crude proteins were analyzed on SDS gel. From left to right: Marker (M), reference standard (2 μg) (RS), induction obtained at 0 hr (I₀), induction obtained at 1 hr (I₁), induction obtained at 2 hr (12), induction obtained at 3 hr (I₃), induction obtained at 4 hr (14), induction obtained at 5 hr (15). The crude protein concentrations were determined by BCA protein assay.

Protein refolding was performed by processes known in the art. For example, as described in U.S. Pat. No. 8,106,160 B2, which is incorporated herein by reference.

### Example 5

### Analysis of Purified Protein

The presence of purified Pro hIFN-α2b was determined by reducing SDS-PAGE. The crude protein extract ran through an ionic exchange column to remove large impurities. Afterwards, samples were run against another SDS-PAGE performed using method known in the art to determine whether the protein produced matches the size of a standard. The protein concentration was determined by BCA protein assay. As noted in FIG. 3, Marker (M), Pro hIFN-α2b (2 μg) (SA), standard protein (5 μg) (RS) all showed that the sample end product matches the standard. C₁₈ reverse phase HPLC was also conducted, with FIG. 4 being a representative curve.

### Example 6

### Yield Comparison

The new methods were run several times as each lot might have slightly different yield. The results showed that an average of 8.13 gram/5 L of fermenter of Pro-hIFN-α2b was obtained. In comparing with method known in the art, this represents a significant increase from 2.12 g to 8.13 g in a 5 L fermenter—an about 383% increase in yield. This increase, much less the significant degree of, is surprising and unexpected due to increased number of steps, which should decrease yield in each additional step. This result cannot be achieved by routine optimization.

### Example 7

### 40 L Fermenter Scale-Up

In this example, a scaled up 40 L fermenter was used as compared Example 3. To scale up, the amounts of all ingredients were increased proportionally without changing the final concentration, in this case, eight folds more than what Example 3 disclosed. However, the fermentation conditions were not changed, for example, carbon and nitrogen source feedings.

More particularly, a vial of recombinant E. coli BLR (DE3) with Methionine-Proline N-terminally modified IFNa-2b gene vector already been transfected by methods known in the art was taken from liquid nitrogen. After thawing and equilibration to room temperature, E. coli cells was transferred into 250 ml of the SYN medium in a sterile 1 L flask. The medium was pre-mixed with about 50 μg/ml kanamycin and about 50 μg/ml chloramphenicol. Cells were then cultured at 37° C. in a shaker incubator at about 100-200 rpm for about 10 to 20 hours of culture. In particular, about 14±3 h or 16±1 h overnight culture.

After cell growth, about 200 to 230 ml, such as about 205, 210 or 220 ml of the starting culture was separately transferred into sterile 250 ml seeding bottles in a laminar flow hood and then pumped into 40 L fermenter(s), with basic medium supplemented with about 1-3 g/L isoleucine, such as about 1.4, 2.0, or 2.6 g/L isoleucine, about 5-10 g/L basic glucose, about 25 μg/ml kanamycin, and about 25 μg/ml chloramphenicol and polarized at about 36-38, such as 36.5 or 37° C. for overnight. The culture was then fed into a fermenter and inoculated for about 3-5 minutes, which is considered as the zero-time-point for measuring cell growth. During fermentation, about 10-20 ml of the culture broth was individually taken at about 1.0-2.0 h interval and subjected to absorption spectrometer to measure optical density at 600 nm. The pH of the fed-batch fermentation was controlled at about pH 7.0-7.5, such as about pH 7.1, 7.2, or 7.5 by automated addition of NH₄OH. The dissolved oxygen (DO) level was controlled to be at less than about 30.0-35.0% (but not equal to 35.0%) of air saturation as the agitation rate gradually increased from about 300 to about 1,000 rpm. Pure oxygen was supplied when agitation speed reached its upper limit, and more oxygen was required. Glucose solution was added using a program-controlled pump that was set to feed when (1) DO level exceeded (or spike above) about 30-40% and agitation rate exceeded about 350-450 rpm; and/or (2) when agitation rate exceeded about 500-600 rpm and pH exceeded about 7.0-7.2. About 30-50 ml/L of antifoam Y-30 emulsion was added to prevent from over-foaming during the fermentation. About 400-480 ml of feeding yeast extract solution was pumped into the fermenter after initial inoculation (seed) at about 5.0, 6.0, 7.0, or 8.0 hours (or S₅, S₆, S₇ or S₈) post seeding depending on OD₆₀₀ or overall fermentation conditions. The expression of protein was achieved by adding about 35-45 ml of about 0.75, 1.0, or 1.5 M IPTG (also known as initial induction, or I₀) into the fermenter along with about 900-1,000 ml of feeding yeast extract solution when the OD₆₀₀ of the fed-batch culture reached a value of about 70-80, for example, about 71, 73, 75, 77, 79, or 80. The exact amount depends on OD₆₀₀ and overall fermentation conditions. After induction, the optical density of the fed-batch culture was taken every hour and recorded.

After about 4-8 hours of IPTG induction, (I₄ to I₈) the fed-batch culture was harvested. The cell broth was collected in centrifugation bottles and centrifuged at about 7,000-9,000 rpm for about 10-20 minutes at 4° C. Supernatants were then discarded and the weights of cell pellets, namely the wet cell weight (WCW), were recorded. The expression level of protein was determined by reducing SDS-PAGE using 4-12% NuPAGE slab gels. The cell pellets were then transferred into a storage box.

The cell pellets obtained from the cell harvest step can be processed in batches or at once. Here, the pellets were resuspended completely in 20 mM TEN buffer (pH=7.0±0.5) with about 7,000 and about 12,000 rpm stirring using a stainless-steel bar in a ratio of about 1 g wet cell pellet to about 1.0, 3.0, or 5.0 ml TEN buffer. Following complete resuspension, the cell pellet/TEN buffer mixture was homogenized twice at about 800-1,200 bar, particularly at about 950, 1,000, 1,050, or 1,100 bar with a homogenizer. The cell mixture was then centrifuged at about 20,000-30,000 rpm for about 30-50 ml/min in the cooling coil and subsequently holding at about 20,000-30,000 rpm for about 10-30 min. The pellet was collected after centrifugation.

The cell pellets were washed with 20 mM TEN buffer (pH=7.0±0.5) at the ratio of about 0.8-1.2 g wet cell pellet to about 1.0 and 3 ml TEN buffer. After washing, the cell pellets were fed to a centrifuge at a feeding rate about 30-40 ml/min with centrifugation speed at about 25,000 rpm for about 40-45 minutes in the cooling coil and subsequently holding at about 20,000-25,000 rpm for about 10-15 min. Supernatants were discarded after centrifugation.

The cell pellets were then mixed with about 4-6 M GnHCI (pH=7.0±0.5) for about 30 minutes to remove unwanted proteins. For each about 2.0-2.5 g wet cell pellet, about 0.5-1.0 ml of about 4-6 M of GnHCI was added. After thorough mixing using a stainless-steel bar, the solution was transferred into a container with the quantity recorded. The solution was fed to a centrifuge at a feeding rate about 20-30 ml/min with about 20,000-25,000 rpm for about 10-20 minutes in the cooling coil and subsequently holding at about 20,000-25,000 rpm for about 20-30 min. Supernatants were discarded after centrifugation and doubly washed inclusion bodies were collected.

The above washed inclusion bodies were then mixed with about 4.0-6.0 M GdnHCI to solubilize the inclusion bodies. For each about 2.0-2.5 g wet cell pellet, about 4-8 ml GnHCI was added. After complete mixing, the solution was transferred into a plastic bottle; and a designated amount of about 0.5 to 2.0 M Dithiothreitol (DTT) was then added to make a final DTT concentration of about 4.0-6.0 mM. The sample mixture was then stirred at a stirring speed of about 200 rpm at room temperature for about 1.0-3.0 hours. Following stirring, the solution was transferred back into centrifuge. The supernatants containing protein were pooled and collected.

Protein refolding was also performed by processes described in U.S. Pat. No. 8,106,160 B2, and the SDS PAGE assay at the 40 L scale showed substantial the same results as compared to Example 5 and/or FIG. 4 of the present disclosure.

The average yield of 40 L fermentation runs was about 24-26 g. As compared to Example 1, this yield is about 1132% to about 1226% increase. More importantly, based on theoretical yield of scaling up, assuming the best ideal scenario of linear increase in yield (which is not the scientific consensus), 5 L to 40 L scale up might have led to a yield increase from 2.12 g to 16.96 g (8 times based on 5 L to 40 L change). However, surprisingly and advantageously, the data show a yield of about 24-26 g on average. This represents about 141% to about 155% increase. It is known in the art that, as the degree of scaling up increases, the yield not only does not increase linearly, it exhibits a curvature decrease with an increase in size. Thus, the yield of the 40 L scale-up demonstrates a surprising, unexpected and unpredictable result based on what is known in the art.

### Example 8

### GCSF

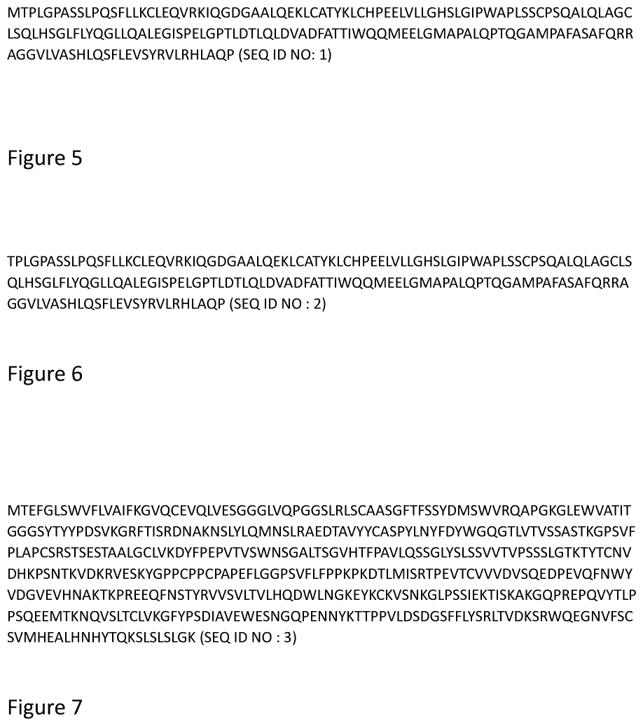

Similarly, the present fermentation method can be applied to produce various proteins. One example is making of GCSF. As GCSF can be fermented in E. Coli, a Methionine (“M”) is typically added at the N terminus. The amino acid sequence of such protein is listed in SEQ ID NO: 1 and in FIG. 5.

Here, a first nucleic acid encoding GCSF can be amplified by PCR using human genomic DNA as a template. The PCR primers can be designed based on the flanking sequences of the coding region of the GCSF gene. The PCR product thus can be cloned into pGEM-T vector and then subcloned into pET-24a, a protein expression vector.

pET-24a vectors, carrying the GCSF genes, can be then transformed into E. coli BL21 or E. coli BLR-CodonPlus (DE3) with RIL vector strain. E. coli clones expressing high levels of the proteins can be selected.

E. coli cells can be transferred into 250 ml of the SYN medium in a sterile 1 L flask. The medium can be pre-mixed with about 50 μg/ml kanamycin and about 50 g/ml chloramphenicol. Cells can be then cultured at about 37° C. in a shaker incubator at about 100-300 rpm for about 10 to 20 hours of culture, in particular, about 16±1 h overnight culture.

After cell growth, about 200 to 230 ml, such as about 205, 210 or 220 ml of the starting culture can be separately transferred into a sterile 250 ml seeding bottles in a laminar flow hood and then can be pumped into fermenters. Initially, each fermenter contains about 1-3 L, basic medium supplemented with about 1-3 g/L isoleucine, such as about 1.4, 2, or 2.6 g/L isoleucine, about 10 g/L basic glucose, about 25 μg/ml kanamycin, and about 25 μg/ml chloramphenicol and polarized at about 36-38° C., such as 36.5 or 37° C. for overnight. The culture can be then fed into a fermenter and inoculate for about 2-4 minutes, which is considered as the zero-time-point for measuring cell growth. During fermentation, about 20 ml of the culture broth can be individually taken at about every 1.5-2 h interval and subjected to absorption spectrometer to measure optical density at 600 nm. The pH of the fed-batch fermentation can be controlled at about pH 7, such as about pH 7.1, 7.2, or 7.5 by automated addition of NH₄OH. The dissolved oxygen (DO) level can be maintained at less than about 35% (not including 35%) of air saturation as the agitation rate gradually can be increased from about 300 to about 1,000 rpm and by supplying pure oxygen when agitation speed reaches its upper limit, and more oxygen is required. Glucose solution can be added using a program-controlled pump that can be set to feed when DO level exceeded about 35-40% and agitation rate exceeds about 300-450 rpm; and/or when agitation rate exceeds about 500-600 rpm and pH exceeded about 7.2. About 50 ml/L of antifoam Y-30 emulsion can be added to prevent from over-foaming during the fed-batch fermentation. About 50-60 ml of feeding yeast extract solution can be pumped into the fermenter after initial inoculated (seed) at about 5, 6, 7, or 8 hours (or S₅, S₆, S₇ or S₈) post seeding depending on OD₆₀₀ or overall fermentation conditions. The expression of protein can be achieved by addition about 5 ml of about 0.75, 1, or 1.5 M IPTG (also known as initial induction, or I₀) into the fermenter along with about 120 ml of feeding yeast extract solution when the OD₆₀₀ of the fed-batch culture reaches a value of about 70-80, for example, about 71, 73, 75, 77, 79, or 80. The exact amount depends on OD₆₀₀ and overall fermentation conditions. After induction, the optical density of the fed-batch culture can be taken every hour and recorded.

After about 4-8 hours of IPTG induction, (I₄ to 18) the fed-batch culture can be harvested. The cell broth can be collected in about a 500 ml centrifugation bottle and centrifuged at about 7,000-9,000 rpm for about 10-20 minutes at 4° C. Supernatant can then be discarded and the weight of cell pellet, namely the wet cell weight (WCW), can be recorded. The expression level of protein can be determined by reducing SDS-PAGE using 4-12% NuPAGE slab gels. The cell pellet can then be transferred into a storage box and stored.

The cell pellets can be obtained from cell harvest step can be resuspended in 20 mM TEN buffer (pH=7.0±0.5) with about 7,000 and about 12,000 rpm stirring using a stainless-steel bar in a ratio of about 1 g wet cell pellet to about 1, 3 or 5 ml TEN buffer. Following complete resuspension, the cell pellet/TEN buffer mixture can be homogenized twice at about 800-1,200 bar, particularly at 950, 1,000, 1,050, or 1,100 bar with a homogenizer. The cell mixture can be then centrifuged by continuous-flow CEPA LE GP at about 20,000-30,000 rpm with a feeding of materials needing to be centrifuged again at a rate about 30-50 ml/min in the cooling coil and subsequently can be held at about 20,000-30,000 rpm for about 10-30 min. The pellet can be collected after centrifugation.

The cell pellets can be washed with 20 mM TEN buffer (pH=7.0±0.5) at the ratio of 1 g wet cell pellet to about 1 and 3 ml TEN buffer. After complete washing, the cell pellets can be fed to centrifuge at a feeding rate 40 ml/min with centrifugation speed at about 25,000 rpm for about 30-42 minutes in the cooling coil and subsequently be held at about 20,000-25,000 rpm for about 15 min. Supernatants can be discarded after continuous-flow centrifugation.

The cell pellets can be then be mixed with 4 M GnHCI (pH=7.0±0.5) for 30 minutes to remove unwanted proteins. For each about 2.5 g wet cell pellet, about 0.5-1 ml of about 3-5 M of GnHCI can be added. After thorough mixing using a stainless-steel bar, the solution can be transferred into 1 L beaker with the quantity recorded. The solution can be fed to centrifuge at a feeding rate about 20-30 ml/min with about 20,000-25,000 rpm for about 10-20 minutes in the cooling coil and subsequently be held at about 20,000-25,000 rpm for about 20-30 min. Supernatants can be discarded after centrifugation and doubly washed inclusion body was collected.

The above inclusion body can then be mixed with about 4-6 M GdnHCI to solubilize the inclusion bodies. For each about every 1.5-2.5 g wet cell pellet, about 0.5-1 ml GnHCI can be added. After complete mixing, the solution can be transferred into a plastic bottle; and a designated amount of about 0.5 to 2 M Dithiothreitol (DTT) can be then added to make a final DTT concentration of about 4-6 mM. The sample mixture can be stirred at a stirring speed of about 200 rpm at room temperature for about 1.5 hours. Following about 1.5 hours of stirring, the dark brown solution can be transferred back into the 500 ml centrifuge tubes. The supernatants containing GCSF can be pooled and collected in the labeled plastic bottle. Typically, the starting Methionine can be removed enzymatically, yielding the end GSCF protein with amino acid sequence denoted in FIG. 6 as SEQ ID NO: 2.

As noted in above Examples, post IPTG induction cell lysates crude proteins can be analyzed on SDS gel. The crude protein concentrations w can be determined by BCA protein assay. Protein refolding can be performed by processes known in the art.

The protein concentration can be determined by BCA protein assay. Significant yield increase of at least about 300% or more can be observed as well.

### Example 9

### mAb Against PD-1

The present fermentation method can be applied to produce antibodies. One example is for producing a monoclonal antibody against PD-1, also known as human cell surface receptor programmed cell death 1.

Here, a first nucleic acid encoding desired monoclonal antibody can be amplified by PCR using human genomic DNA as a template. The PCR primers can be designed based on the flanking sequences of the coding region of the monoclonal antibody gene. The PCR product thus can be cloned into pGEM-T vector and then subcloned into pET-24a, a protein expression vector. The amino acid sequence for the protein to be cloned is denoted in FIG. 7 as SEQ ID NO: 3.

pET-24a vectors, carrying the monoclonal antibody genes, can be then transformed into E. coli BL21, E. coli BLR-CodonPlus (DE3), or E. coli BLR21 (DE3)-RIL strain. E. coli clones expressing high levels of these two proteins can be selected.

E. coli cells can be transferred into 250 ml of the SYN medium in a sterile 1 L flask. The medium can be pre-mixed with about 30-60 μg/ml kanamycin and about 50 g/ml chloramphenicol. Cells can be then cultured at about 36-37° C. in a shaker incubator at about 200 rpm for about 10 to 20 hours of culture, in particular, about 16±1 h overnight culture.

After cell growth, about 200 to 230 ml, such as about 205, 210 or 220 ml of the starting culture can be separately transferred into a sterile 250 ml seeding bottles in and then can be pumped into fermenters. Initially, each fermenter contains about 1-3 L, basic medium supplemented with about 1-3 g/L isoleucine, such as about 1.4, 2, or 2.6 g/L isoleucine, about 10 g/L basic glucose, about 25 μg/ml kanamycin, and about 25 μg/ml chloramphenicol and polarized at about 36-38, such as 36.5 or 37° C. for overnight. The culture can be then fed into a fermenter and inoculate for about 3 minutes, which is considered as the zero-time-point for measuring cell growth. During fermentation, about 20 ml of the culture broth can be individually taken at about 2 h interval and subjected to absorption spectrometer to measure optical density at 600 nm. The pH of the fed-batch fermentation can be controlled at about pH 7, such as about pH 7.1, 7.2, or 7.5 by automated addition of NH₄OH. The dissolved oxygen (DO) level can be maintained at less than about 25-40% (not including 35%) of air saturation as the agitation rate gradually can be increased from about 300 to about 1,000 rpm and by supplying pure oxygen when agitation speed reaches its upper limit, and more oxygen is required. Glucose solution can be added using a program-controlled pump that can be set to feed when DO level exceeded about 30-40% and agitation rate exceeds about 300-450 rpm; and/or when agitation rate exceeds about 600 rpm and pH exceeded about 7.2. About 50 ml/L of antifoam Y-30 emulsion can be added to prevent from over-foaming during the fed-batch fermentation. About 50-60 ml of feeding yeast extract solution can be pumped into the fermenter after initial inoculated (seed) at about 5, 6, 7, or 8 hours (or S₅, S₆, S₇ or S₈) post seeding depending on OD₆₀₀ or overall fermentation conditions. The expression of protein can be achieved by addition about 3 to 5 ml of about 0.70 to 0.85, 1, or 1.5 M IPTG (also known as initial induction, or I₀) into the fermenter along with about 120 ml of feeding yeast extract solution when the OD₆₀₀ of the fed-batch culture reaches a value of about 70-80, for example, about 71, 73, 75, 77, 79, or 80. The exact amount depends on OD₆₀₀ and overall fermentation conditions. After induction, the optical density of the fed-batch culture can be taken every hour and recorded.

After about 4-8 hours of IPTG induction, (I₄ to 18) the fed-batch culture can be harvested. The cell broth can be collected in about a 500 ml centrifugation bottle and centrifuged at about 7,000-9,000 rpm for about 10-20 minutes at 4° C. Supernatant can then be discarded and the weight of cell pellet, namely the wet cell weight (WCW), can be recorded. The expression level of protein can be determined by reducing SDS-PAGE using 4-12% NuPAGE slab gels. The cell pellet can then be transferred into a storage box and stored.

The cell pellets can be obtained from cell harvest step can be resuspended in about 15-20 mM TEN buffer (pH=7.0±0.5) with about 7,000 and about 12,000 rpm stirring using a stainless-steel bar in a ratio of about 1 g wet cell pellet to about 1, 3 or 5 ml TEN buffer. Following complete resuspension, the cell pellet/TEN buffer mixture can be homogenized twice at about 800-1,200 bar, particularly at 950, 1,000, 1,050, or 1,100 bar with a homogenizer. The cell mixture can be then centrifuged by continuous-flow CEPA LE GP at about 20,000-30,000 rpm with a feeding of materials needing to be centrifuged again at a rate about 30-50 ml/min in the cooling coil and subsequently can be held at about 20,000-30,000 rpm for about 10-30 min. The pellet can be collected after centrifugation.

The cell pellets can be washed with 20 mM TEN buffer (pH=7.0±0.5) at the ratio of 1 g wet cell pellet to 1 and 3 ml TEN buffer. After complete washing, the cell pellets can be fed to centrifuge at a feeding rate 40 ml/min with centrifugation speed at about 25,000 rpm for about 30-42 minutes in the cooling coil and subsequently be held at about 20,000-25,000 rpm for about 15 min. Supernatants can be discarded after continuous-flow centrifugation.

The cell pellets can be then be mixed with 4 M GnHCI (pH=7.0±0.5) for 30 minutes to remove unwanted proteins. For each about 2.5 g wet cell pellet, about 0.5-1 ml of about 3-5 M of GnHCI can be added. After thorough mixing using a stainless-steel bar, the solution can be transferred into 1 L beaker with the quantity recorded. The solution can be fed to centrifuge at a feeding rate about 20-30 ml/min with about 20,000-25,000 rpm for about 10-20 minutes in the cooling coil and subsequently be held at about 20,000-25,000 rpm for about 20-30 min. Supernatants can be discarded after centrifugation and doubly washed inclusion body was collected.

The above inclusion body can then be mixed with about 4-6 M GdnHCI to solubilize the inclusion bodies. For each about every 1.5-2.5 g wet cell pellet, about 0.5-1 ml GnHCI can be added. After complete mixing, the solution can be transferred into a plastic bottle; and a designated amount of about 0.5 to 2 M Dithiothreitol (DTT) can be then added to make a final DTT concentration of about 4-6 mM. The sample mixture can be stirred at a stirring speed of about 200 rpm at room temperature for about 1.5 hours. Following about 1.5 hours of stirring, the dark brown solution can be transferred back into the 500 ml centrifuge tubes. The supernatants containing anti-PD1 antibody can be pooled and collected in the labeled plastic bottle.

As noted in above Examples, post IPTG induction cell lysates crude proteins can be analyzed on SDS gel. The crude protein concentrations w can be determined by BCA protein assay. Protein refolding can be performed by processes known in the art.

The protein concentration can be determined by BCA protein assay. Significant yield increase of at least about 200-600% or more can be observed as well.

It is contemplated that any embodiment discussed in this specification can be implemented with respect to any method, kit, reagent, or composition of the disclosure, and vice versa. Furthermore, compositions of the disclosure can be used to achieve methods of the disclosure.

It will be understood that the particular embodiments described herein are shown by way of illustration and not as limitations of the disclosure. The principal features of this disclosure can be employed in various embodiments without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of certain embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the disclosure as defined by the appended claims.

What is claimed is:

1. A method to increase recombinant protein production in a prokaryote host cell, comprising:
performing a recombinant protein fermentation including the steps of:
carrying out seeding (time S₀) of a culture of prokaryote host cell engineered to inducibly express a recombinant protein and transferring the culture into one or more fermenters each containing a culture medium;
measuring a dissolved oxygen (DO) level, an agitation rate, and a pH in the one or more fermenters;
supplying a carbon source feeding solution to any of the one or more fermenters individually, whenever condition(s) (i) and/or (ii) are met:
(i) said DO level exceeds above about 35% to 45% and said agitation rate exceeds about 300 to 1,000 rpm;
(ii) said agitation rate exceeds about 400 to 700 rpm and said pH exceeds about 7.0 to 7.4; and
supplying a nitrogen source to any of the one or more fermenters in which induction of expression of the recombinant protein has been initiated, individually, at about I₀ (time at initiation of induction) or I₁ or both;
optionally, supplying a nitrogen source to any of the one or more fermenters, individually, at one or more time points selected from about S₅, S₆, S₇, S₈, S₉ and S₁₀.

2. The method according to claim 1, wherein (i) said DO level exceeds above about 35% to 40% and said agitation rate exceeds about 300 to 700 rpm.

3. The method according to claim 1, wherein (ii) said agitation rate exceeds about 500 to 600 rpm and said pH exceeds about 7.0 to 7.4.

4. The method according to claim 1, wherein the nitrogen source is supplied, individually, at about I₀ or I₁ or both, and at one or more time points selected from about S₅, S₆, S₇, S₈, S₉ and S₁₀.

5. The method according to claim 1, wherein a carbon source feeding solution is supplied at least once to at least one fermenter during the recombinant protein fermentation; optionally, the carbon source feeding solution is a glucose feeding solution, fructose feeding solution, galactose feeding solution, pyruvate feeding solution, or any combination thereof.

6. The method according to claim 5, wherein a cumulative total of about 800 to 1,500 g of glucose is supplied to at least one fermenter, per about 5 L fermenter capacity, by the end of the recombinant protein fermentation.

7. The method according to claim 1, wherein the nitrogen source supplied to the one or more fermenters, individually, comprises potassium.

8. The method according to claim 1, wherein the nitrogen source supplied to the one or more fermenters, individually, comprises a yeast extract, peptone, soytone, urea, tryptone, or any combinations thereof.

9. The method of claim 8, wherein about 40 to 150 mL of a yeast extract solution is supplied the one or more fermenters each time, individually; optionally about 300 to 1,500 mL of a yeast extract solution is supplied to the one or more fermenters each time, individually.

10. The method according to claim 1, wherein the recombinant protein fermentation is performed with an inoculum ratio of about 6.0 to 8.5%.

11. The method according to claim 1, wherein said recombinant protein comprises VX-001, AVE-9633, AC-9301, NY-ESO-1 vaccine, erythropoietin, EPO-Fc, CNTO-528, AMG-114, JR-013, Factor XIII, IFN-α2b, Pro-IFN-α2b, anti-PD1 antibody, and/or GCSF.

12. The method according to claim 1, wherein said prokaryote host cell comprises an E. coli BLR-Codon Plus (DE3) cell.

13. The method according to claim 1, further comprising a step of collecting the culture of prokaryote host cell or a sample of the culture in the one or more fermenters after induction of expression of the recombinant protein, individually.

14. The method according to claim 13, further comprising processing inclusion bodies in the collected culture of prokaryote cell or sample to obtain a crude protein extract.

15. The method according to claim 13, wherein the culture or sample are collected after or at about I₄ to I₈.

16. The method according to claim 14, further comprising determining yield of the expressed recombinant protein in the crude protein extract, wherein the yield is increased by at least about 5%, at least 10%, at least 15%, at least 20%, at least 25%, at least 30%, at least 35%, at least 40%, at least 45%, at least 50%, at least 55%, at least 60%, at least about 62.7%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 167%, at least 180%, at least 190%, at least 200%, at least 210%, at least 220%, at least 230%, at least 240%, at least 250%, at least 260%, at least 270%, at least 280%, at least 290%, at least 300%, at least 350%, at least 383%, at least 400%, at least 450%, at least 500%, at least 550%, at least 600%, at least 650%, at least 700%, at least 748%, at least 750%, at least 800%, at least 9 folds, at least 10 folds, at least 20 folds or more, as compared to another fermentation method.

17. The method according to claim 16, wherein the yield is determined for a fermenter having about a 40 L capacity and the yield is increased by at least about 100% to 200%, 100%, 105%, 110%, 115%, 120%, 125%, 130%, 135%, 140%, 145%, 150%, 155%, 160%, 165%, 170%, 175%, 180%, 190%, or 200%, as compared to the yield of a fermenter having about a 5 L capacity in the other fermentation method.

18. The method according to claim 14, further comprising determining yield of the expressed recombinant protein in the crude protein extract, wherein the yield is about 5 to 10 g of the recombinant protein in a fermenter having about a 5 L capacity; optionally, wherein the yield is about 20 to 30 g of the recombinant protein in a fermenter having about a 40 L capacity.

19. A program-controlled mechanism or equipment comprising:
a. an apparatus equipped with one or more sensors capable of measuring the oxygen level, pH, and/or agitation of culture medium in a fermentation reactor in a fermentation process in a periodic or continuous mode; and
b. a feeding solution injection system integrated within said program-controlled mechanism that is programmed to provide one or more feeding solutions to a fermentation reactor based on measured oxygen levels, pH and/or agitation in the culture medium in a periodic or continuous interval.

20. A method for automatically regulating glucose concentration, oxygen level, pH and/or agitation during fermentation, comprising:
a. measuring parameters of oxygen level, pH and/or agitation of the fermentation with one or more sensors;
b. transmitting the measured parameters to a program-controlled mechanism; and
c. automatically adjusting the injection rate, volume of a feeding solution and agitation based on the measured parameters.


SEQUENCE LISTING (3 sequences)

SEQ ID NO: 1 - protein, 175 aa, Homo sapiens
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - protein, 174 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 3 - protein, 464 aa, Homo sapiens
MTEFGLSWVFLVAIFKGVQCEVQLVESGGGLVQPGGSLRLSCAASGFTFSSYDMSWVRQAPGKGLEWVATITGGGSYTYYPDSVKGRFTISRDNAKNSLYLQMNSLRAEDTAVYYCASPYLNYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK